(19) United States
(12) Patent Application Publication
(10) Pub. No.: US 2017/0369519 A1
(43) Pub. Date: Dec. 28, 2017

(54) METHODS FOR THE STEREOSELECTIVE PREPARATION OF APIOSE DERIVATIVES FROM ALLYLIC ALCOHOL COMPOUNDS AND ALLENE COMPOUNDS USING CATALYTIC ASYMMETRIC SYNTHESIS

(71) Applicant: POSTECH ACADEMY-INDUSTRY FOUNDATION, Pohang (KR)

(72) Inventors: Young Ho RHEE, Pohang (KR); Mijin KIM, Suwon (KR); Soyeong KANG, Pohang (KR)

(21) Appl. No.: 15/488,603

(22) Filed: Apr. 17, 2017

(30) Foreign Application Priority Data
Jun. 27, 2016 (KR) 10-2016-0079958

(51) Int. Cl. C07H 17/04; C07H 15/04; C07H 3/06; C07H 3/04; C07D 407/12; C07D 309/10; C07D 307/32; C07H 15/207; C07C 43/215

(52) U.S. Cl. CPC C07H 17/04; C07H 15/207; C07H 15/04; C07H 3/06; C07C 43/215; C07D 407/12; C07D 309/10; C07D 307/32; C07H 3/04

(57) ABSTRACT

The present invention relates to a method for the stereoselective preparation of apiose derivatives from allylic alcohol compounds and allene compounds using catalytic asymmetric synthesis. The method for the stereoselective preparation of apiose derivatives of the present invention is based on the catalytic asymmetric synthesis from allylic alcohol compounds and allene compounds in the presence of a metal catalyst, so that apiose derivatives can be produced stereoselectively, with high yield, with high optical purity regardless of the types of substituents of the compounds. The method of the invention can also be used for the preparation of oligosaccharides including monosaccharides, disaccharides, and polysaccharides or various compounds including apiose derivatives because the method can minimize the production of by-products without using an activating group, unlike the conventional method for the preparation of adipose derivatives.

### BACKGROUND OF THE INVENTION

### 1. Field of the Invention

The present invention relates to a method for the stereoselective preparation of apiose derivatives from allylic alcohol compounds and allene compounds using catalytic asymmetric synthesis.

### 2. Description of the Related Art

The stereoselective synthesis of carbohydrates with high chemical efficiency is a major target in the field of organic synthetic chemistry.

Apiose is one of furanose sugars with unique side chains having natural (3R)-stereochemical forms, which is found in various natural substances. Apiose also exists as a terminal or internal residue of various polysaccharides. In particular, the apiofuranose-containing glycosides such as saponin, flavonoid and phenol glycoside which are rich in natural substances have been confirmed to have a biological activity (non-patent reference 1).

[Apiose-Containing Oligosaccharides]

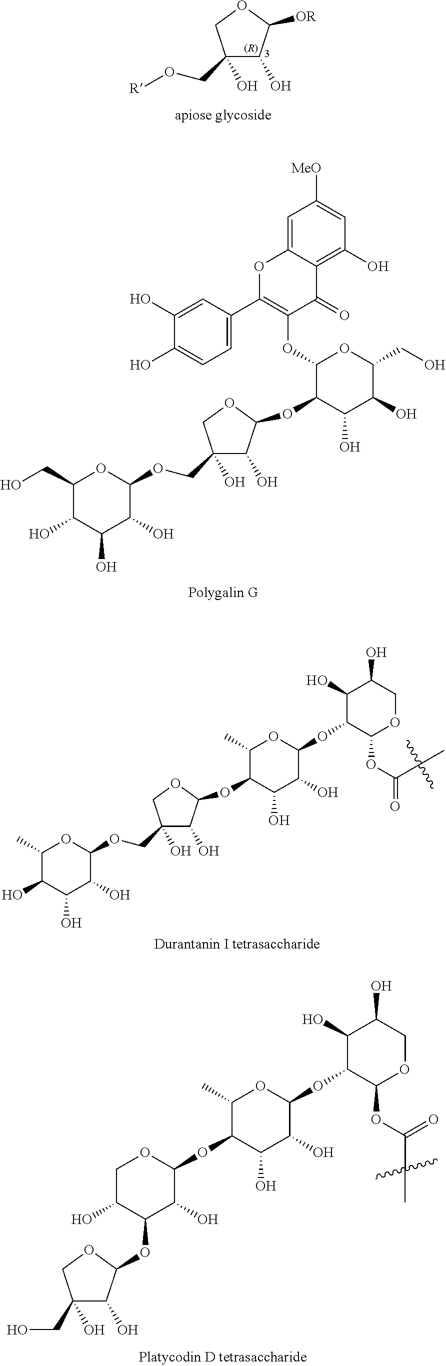

Thus, studies on synthesis of apiose-containing oligosaccharides have been actively going on.

According to the conventional methods, the introduction of apiose residue in oligosaccharides depends on linear synthesis based on binding between an activated furanose precursor (glycosyl donor) and an alcohol moiety (glycosyl acceptor).

[Reaction Formula of Conventional Method]

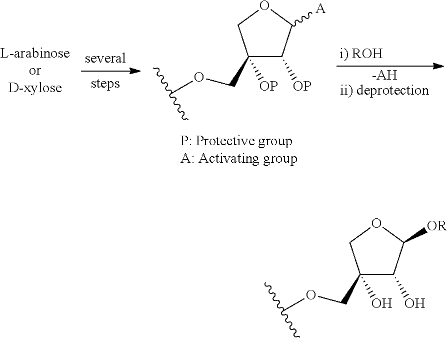

In order to synthesis a furanose precursor by the conventional synthesis method, multiple steps are required and a direct protective group such as acetyl or benzylidene group (P in reaction formula of the conventional synthesis method) is required to obtain a target compound displaying a clear stereochemical structure observed in all of natural substances. Also, an activating group (A in reaction formula of the conventional synthesis method) that has to be eliminated after the glycosylation is necessary for the reaction in the conventional method.

The new approach based on the idea that glycosidic bond is formed by asymmetric reaction is considered as a most promising alternative of glycosylation (non-patent reference 2). However, this new approach is only limited in the synthesis of pyranose.

The present inventors have studied to prepare apiose derivatives, particularly an oligosaccharide compound containing glycosidic bond. In the course of the study, the present inventors confirmed that the method for the preparation of apiose derivatives of the present invention was efficient in preparing apiose derivatives from allylic alcohol compounds and allene compounds since the method did not require an activating group because it was based on catalytic asymmetric synthesis; could minimize the production of by-products; and was composed of relative shorter procedure with less steps, leading to the completion of the present invention.

### PRIOR ART REFERENCE

### Non-Patent Reference

Nat. Prod. Res. 2013, 27, 1220-1227

Chem. Commun. 2015, 51, 17475-17478

### SUMMARY OF THE INVENTION

It is an object of the present invention to provide a method for the stereoselective preparation of apiose derivatives.

It is another object of the present invention to provide an intermediate compound for the preparation of apiose derivatives.

To achieve the above objects, the present invention provides a method for the stereoselective preparation of apiose derivatives comprising the following steps as shown in reaction formula 1:

preparing the acyclic acetal compound represented by formula 3 by reacting the allylic alcohol compound represented by formula 1 with the allene compound represented by formula 2 in the presence of a metal catalyst (step 1);

preparing the cyclic acetal compound represented by formula 4 by inducing ring closing metathesis of the acyclic acetal compound represented by formula 3 obtained in step 1 in the presence of a metal catalyst (step 2); and

preparing the compound represented by formula 5 by reacting the compound represented by formula 4 obtained in step 2 in the presence of a metal catalyst (step 3).

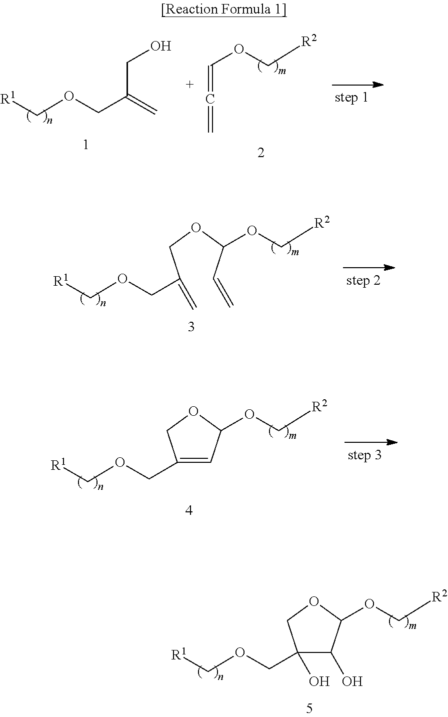

The present invention also provides an intermediate compound for the stereoselective preparation of apiose derivatives represented by formula 3 below:

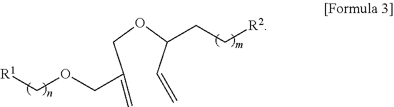

In addition, the present invention provides an intermediate compound for the stereoselective preparation of apiose derivatives represented by formula 4 below:

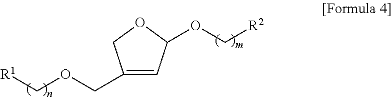

### Advantageous Effect

The method for the stereoselective preparation of apiose derivatives of the present invention is efficient in preparing apiose derivatives from allylic alcohol compounds and allene compounds in the presence of a metal catalyst stereoselectively with high yield and high optical purity, regardless of the kinds of substituents of the compound, by using catalytic asymmetric synthesis. The method of the invention can also be used for the preparation of oligosaccharides including monosaccharides, disaccharides, and polysaccharides or various compounds including apiose derivatives because the method can minimize the production of by-products without using an activating group, unlike the conventional method for the preparation of adipose derivatives.

### DESCRIPTION OF THE PREFERRED EMBODIMENTS

Hereinafter, the present invention is described in detail.

The present invention provides a method for the stereoselective preparation of apiose derivatives comprising the following steps as shown in reaction formula 1:

preparing the acyclic acetal compound represented by formula 3 by reacting the allylic alcohol compound represented by formula 1 with the allene compound represented by formula 2 in the presence of a metal catalyst (step 1);

preparing the cyclic acetal compound represented by formula 4 by inducing ring closing metathesis of the acyclic acetal compound represented by formula 3 obtained in step 1 in the presence of a metal catalyst (step 2); and

preparing the compound represented by formula 5 by reacting the compound represented by formula 4 obtained in step 2 in the presence of a metal catalyst (step 3).

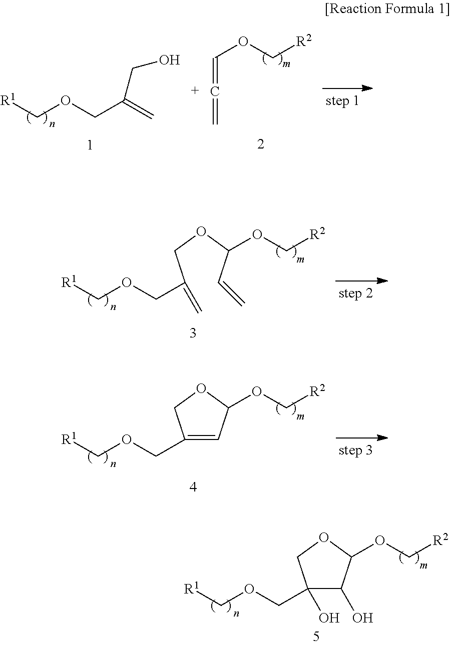

In the reaction formula 1,

R¹ and R² are independently hydrogen; unsubstituted or substituted straight or branched C₁₋₅ alkyl; unsubstituted or substituted straight or branched C₁₋₅ alkoxy; unsubstituted or substituted 3˜8 membered cycloalkyl; unsubstituted or substituted 3˜8 membered heterocycloalkyl containing one or more O atoms; or unsubstituted or substituted 6˜10 membered aryl;

Wherein, the substituted C₁₋₅ alkyl and C₁₋₅ alkoxy can be substituted with one or more substituents selected from the group consisting of halogen, —OH, unsubstituted 6˜10 membered aryl and unsubstituted 6˜10 membered aryloxy, and the substituted cycloalkyl, heterocycloalkyl, and aryl can be substituted with one or more substituents selected from the group consisting of halogen, —OH, —(CH₂)ₚOR³, unsubstituted straight or branched C₁₋₅ alkyl, unsubstituted straight or branched C₁₋₅ alkoxy, unsubstituted 6˜10 membered aryl, unsubstituted 6˜10 membered aryloxy, unsubstituted 6˜10 membered arylcarbonyl,

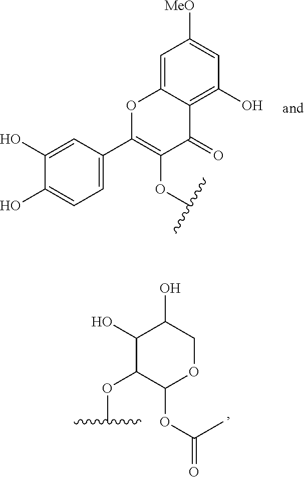 [SMILES: *Oc1c(-c2ccc(O)c(O)c2)oc2cc(OC)cc(O)c2c1=O ; *OC1C(OC(C)=O)OCC(O)C1O]

The said R³ is unsubstituted 6˜10 membered arylC₀₋₂alkyl or unsubstituted 6˜10 membered arylcarbonyl; and

n, m and p are independently 0 or 1.

Hereinafter, the method for the stereoselective preparation of apiose derivatives of the invention is described in more detail.

In the method for the stereoselective preparation of apiose derivatives of the present invention, step 1 is to prepare the acyclic acetal compound represented by formula 3 by reacting the allylic alcohol compound represented by formula 1 with the allene compound represented by formula 2 in the presence of a metal catalyst. Particularly, O,O-acetal is formed in this step from the allylic alcohol compound and the allene compound through hydroalkoxylation in the presence of a metal catalyst and a chiral ligand.

At this time, the metal catalyst is not limited but is preferably a transition metal complex catalyst, which is exemplified by Pd, Ru, Os, Co, Ni, Pt, W, Sn, Al, Mo, and Re transition metal complex, and is more preferably Pd transition metal complex. The Pd transition metal complex is not limited but is preferably Pd₂(dba)₃, Pd(Ph₃P)₄, Pd/C, PdCl₂(PPh₃)₂, PdCl₂ (dppf), [PdCl(allyl)]₂, Pd(OAc)₂, or PdCl₂, and is more preferably Pd₂(dba)₃.

The metal catalyst above is not limited herein. The concentration of such a metal catalyst usable herein is 2˜5 mol % by the volume of the allene compound represented by formula 2. If the concentration of the catalyst is less than 2 mol %, reaction would not be completed and the starting material would remain and the reaction velocity would be slower. If the concentration of the metal catalyst is more than 5 mol %, although the amount is increased, yield would not be any better, compared with the concentration under 5 mol %, suggesting that the extra volume would be wasted. Therefore, it is preferred to add the catalyst at the concentration of not more than mol %.

The chiral ligand above is not limited but is preferably (R,R)-DACH naphthyl trost ligand, (S,S)-DACH naphthyl trost ligand, (R,R)-DACH phenyl trost ligand, or (S,S)-DACH phenyl trost ligand.

The concentration of the chiral ligand above is not limited but is preferably 3˜10 mol % by the total volume of the allene compound represented by formula 2. If the concentration is less than 3 mol %, reaction would not be completed and the starting material would remain and the reaction velocity would be slower. On the other hand, if the concentration is more than 10 mol %, even if the concentration is increased, yield would not be increased anymore compared with the yield when the ligand is added at the concentration of less than 10 mol %. Therefore, it is preferred that the volume of the chiral ligand is preferably not more than 10 mol %.

The apiose derivatives of the present invention can be stereoselectively prepared by using the chiral ligand above.

The ligand dependent stereoselectivity of the stereoselective preparation method for apiose derivatives of the present invention was investigated. As a result, the stereochemical structure was different according to the types of ligand (see Experimental Example 2-1 and Table 4).

In addition, an inorganic base or an organic base can be additionally added in step 1 above.

At this time, the inorganic base is not limited but preferably tripotassium phosphate, dipotassium phosphate, monopotassium phosphate, sodium hydroxide, sodium carbonate, potassium carbonate, cesium carbonate, or sodium hydride. The organic base above is not limited but preferably triethylamine, pyridine, N,N-diisopropylethylamine (DIPEA), or 1,8-diazabicyclo[5.4.0]-7-undecene(DBU), and more preferably triethylamine.

The inorganic base or organic base above can be added to 1 eq of the allene compound represented by formula 2 at the concentration of 0.05˜2 eq, but not always limited thereto. If the concentration of the inorganic base or organic base is less than 0.05 eq, reaction would not be completed and the staring material would remain and the reaction velocity would be slower. On the other hand, if the concentration is more than 2 eq, in spite of the increase of the concentration, yield would not be increased any better, suggesting that there is no advantage, so that the volume is preferably not more than 2 eq.

In the method for the stereoselective preparation of apiose derivatives of the present invention, step 2 is to prepare the cyclic acetal compound represented by formula 4 by inducing ring closing metathesis of the acyclic acetal compound represented by formula 3 obtained in step 1 in the presence of a metal catalyst.

At this time, the metal catalyst is not limited and any general catalyst useable in the ring metathesis is accepted, but is preferably a transition metal complex catalyst, and more preferably such a transition metal complex catalyst Ru, Pd, Os, Co, Ni, Pt, W, Sn, Al, Mo, and Re. Among these, Ru transition metal complex catalyst is most preferred. The said Ru complex catalyst is not limited but is preferably 1^{st} generation Grubbs' catalyst, 2^{nd} generation Grubbs' catalyst, 1^{St} generation Hoveyda-Grubbs' catalyst, or 2^{nd} generation Hoveyda-Grubbs' catalyst, and more preferably 1^{st} generation Grubbs' catalyst.

The concentration of the metal catalyst added to the allene compound represented by formula 2 is not limited but preferably 3˜15 mol %. If the concentration of the catalyst is less than 3 mol %, reaction would not be completed and the starting material would remain. If the concentration is more than 15 mol %, in spite of the increased concentration, there would be no advantage in the yield, compared with the concentration under 15 mol %. Therefore, it is preferred that the concentration of the catalyst is not more than 15 mol %.

The reaction solvent used in step 1 and step 2 is not limited but preferably a non-polar solvent such as toluene, dichloromethane, 1,4-dioxane, hexane, benzene, chloroform, or diethylether, and more preferably toluene, dichloromethane, or 1,4-dioxane.

In the method for the stereoselective preparation of the invention, solvent dependent ee value was measured in step 1. As a result, when dichloromethane or 1,4-dioxane, the more polar solvent, was used, ee value was 92% or 87%. In the meantime, when toluene, the most non-polar solvent, was used, ee value was 96%, which was the highest (see Experimental Example 1-1 and Table 2).

In the method for the stereoselective preparation of apiose derivatives of the present invention, step 3 is to prepare the compound represented by formula 5 by reacting the compound represented by formula 4 prepared in step 2 in the presence of a metal catalyst. Particularly, double bond of the acyclic acetal compound prepared in step 2 was converted into diol via dihydroxylation to form apiose structure therein, resulting in the preparation of apiose derivatives.

At this time, the metal catalyst above is not limited but preferably a transition metal catalyst, more preferably OsO₄, Os or Mn, and most preferably OsO₄. The amount of the catalyst is preferably 0.01˜0.1 eq to 1 eq of the cyclic acetal compound represented by formula 4. If the amount of the catalyst is less than 0.01 eq, reaction would not be completed and the starting material would remain. Even if the amount of the catalyst is more than 0.1 eq, there would be no advantages in the yield, compared with when the amount is less than 0.1 eq. Therefore, it is preferred to use the catalyst not more than 0.1 eq.

The yield of each product in each step of the method of the invention was measured. Precisely, the acyclic acetal compound having a stereochemical structure, which is the goal of the stereoselective preparation, was produced in step 1 through hydroalkoxylation with the yield of 72˜99%. The target cyclic acetal compound having a stereochemical structure was prepared in step 2 through ring closing metathesis (RCM) with the yield of 64˜86%. And at last, the apiose derivative having a stereochemical structure, which is the goal of this step, was prepared in step 3 through cyclic acetal dehydroxylation with the yield of 50˜99.9% (see Examples 1˜11 and Table 1).

Therefore, apiose derivatives, in particular oligosaccharides including monosaccharides, disaccharides, and polysaccharides can be stereoselectively prepared with high yield through simple steps of the method for the stereoselective preparation of apiose derivatives of the present invention.

As described hereinbefore, the method for the stereoselective preparation of apiose derivatives of the present invention is efficient in preparing apiose derivatives from allylic alcohol compounds and allene compounds through catalytic asymmetric synthesis in the presence of a metal catalyst, regardless of the kinds of substituents of the compounds, stereoselectively with high yield and high optical purity. Unlike the conventional method for the preparation of apiose derivatives, the method of the present invention does not require an activating group and can minimize the generation of by-products, so that the method of the invention can be effectively used for the preparation of various compounds containing apiose derivatives, in particular oligosaccharides such as monosaccharides, disaccharides and polysaccharides.

The present invention also provides an intermediate compound for the preparation of apiose derivatives represented by formula 3 below:

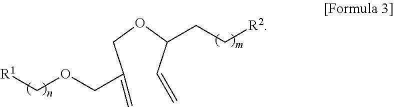

In the formula 3,

R¹ and R² are independently hydrogen; unsubstituted or substituted straight or branched C₁₋₅ alkyl; unsubstituted or substituted straight or branched C₁₋₅ alkoxy; unsubstituted or substituted 3˜8 membered cycloalkyl; unsubstituted or substituted 3˜8 membered heterocycloalkyl containing one or more O atoms; or unsubstituted or substituted 6˜10 membered aryl;

Wherein, the substituted C₁₋₅ alkyl and C₁₋₅ alkoxy can be substituted with one or more substituents selected from the group consisting of halogen, —OH, unsubstituted 6˜10 membered aryl and unsubstituted 6˜10 membered aryloxy, and the substituted cycloalkyl, heterocycloalkyl, and aryl can be substituted with one or more substituents selected from the group consisting of halogen, —OH, —(CH₂)ₚOR³, unsubstituted straight or branched C₁₋₅ alkyl, unsubstituted straight or branched C₁₋₅ alkoxy, unsubstituted 6˜10 membered aryl, unsubstituted 6˜10 membered aryloxy, unsubstituted 6˜10 membered arylcarbonyl,

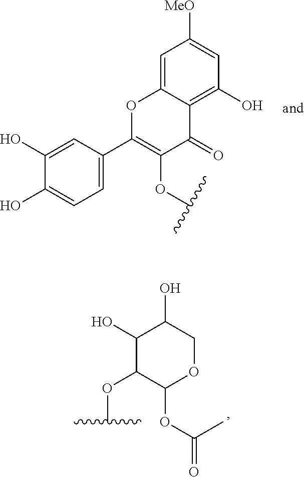 [SMILES: *Oc1c(-c2ccc(O)c(O)c2)oc2cc(OC)cc(O)c2c1=O ; *OC1C(OC(C)=O)OCC(O)C1O]

The said R³ is unsubstituted 6˜10 membered arylC₀₋₂alkyl or unsubstituted 6˜10 membered arylcarbonyl; and

n, m and p are independently 0 or 1.

Preferably,

R¹ and R² are independently unsubstituted or substituted straight or branched C₁₋₃ alkyl; unsubstituted or substituted straight or branched C₁₋₃ alkoxy; unsubstituted or substituted 5˜6 membered cycloalkyl; unsubstituted or substituted 5˜6 membered heterocycloalkyl containing one or more O atoms; or unsubstituted or substituted 6˜8 membered aryl;

Wherein, the substituted C₁₋₃ alkyl and C₁₋₃ alkoxy can be substituted with one or more substituents selected from the group consisting of fluoro, chloro, —OH, unsubstituted phenyl, and unsubstituted phenoxy, and the substituted cycloalkyl, heterocycloalkyl, and aryl can be substituted with one or more substituents selected from the group consisting of fluoro, chloro, —OH, —(CH₂)ₚOR³, methyl, ethyl, propyl, isopropyl, methoxy, ethoxy, propoxy, isopropoxy, unsubstituted phenyl, unsubstituted phenoxy, unsubstituted benzoyl,

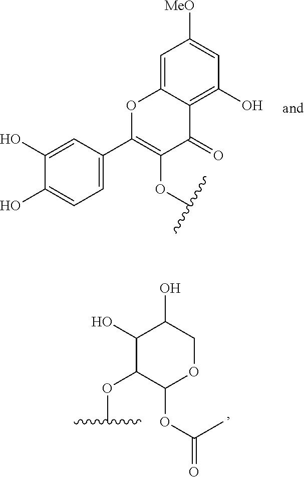 [SMILES: *Oc1c(-c2ccc(O)c(O)c2)oc2cc(OC)cc(O)c2c1=O ; *OC1C(OC(C)=O)OCC(O)C1O]

The said R³ is phenyl, benzyl, or benzoyl; and

n, m and p are independently 0 or 1.

More preferably,

R¹ and R² are independently unsubstituted or substituted cyclohexyl, unsubstituted or substituted tetrahydropyranyl, or unsubstituted or substituted phenyl;

Wherein, the substituted cyclohexyl, tetrahydropyranyl, and phenyl can be substituted with one or more substituents selected from the group consisting of —(CH₂)ₚOR³, methyl, methoxy, isopropoxy,

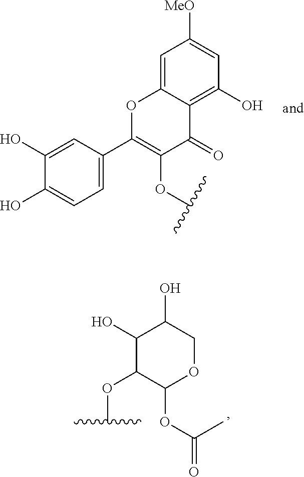 [SMILES: *Oc1c(-c2ccc(O)c(O)c2)oc2cc(OC)cc(O)c2c1=O ; *OC1C(OC(C)=O)OCC(O)C1O]

The said R³ is benzyl or benzoyl; and

n, m and p are independently 0 or 1.

The intermediate compound for the preparation of apiose derivatives represented by formula 3 is produced by performing step 1 of the method for the preparation of apiose derivatives of the present invention, and can be used for the preparation of apiose derivatives through ring closing metathesis and dehydroxylation.

In addition, the present invention provides an intermediate compound for the stereoselective preparation of apiose derivatives represented by formula 4 below:

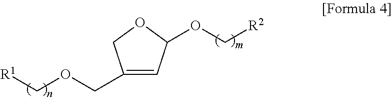

In the formula 4,

R¹ and R² are independently hydrogen; unsubstituted or substituted straight or branched C₁₋₅ alkyl; unsubstituted or substituted straight or branched C₁₋₅ alkoxy; unsubstituted or substituted 3˜8 membered cycloalkyl; unsubstituted or substituted 3˜8 membered heterocycloalkyl containing one or more O atoms; or unsubstituted or substituted 6˜10 membered aryl;

Wherein, the substituted C₁₋₅ alkyl and C₁₋₅ alkoxy can be substituted with one or more substituents selected from the group consisting of halogen, —OH, unsubstituted 6˜10 membered aryl and unsubstituted 6˜10 membered aryloxy, and the substituted cycloalkyl, heterocycloalkyl, and aryl can be substituted with one or more substituents selected from the group consisting of halogen, —OH, —(CH₂)ₚOR³, unsubstituted straight or branched C₁₋₅ alkyl, unsubstituted straight or branched C₁₋₅ alkoxy, unsubstituted 6˜10 membered aryl, unsubstituted 6˜10 membered aryloxy, unsubstituted 6˜10 membered arylcarbonyl,

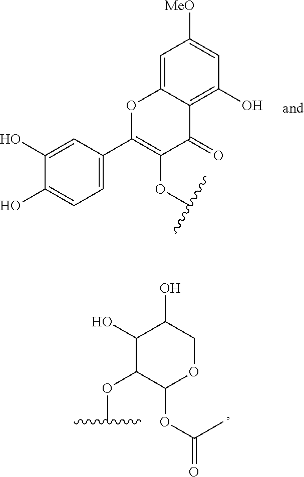 [SMILES: *Oc1c(-c2ccc(O)c(O)c2)oc2cc(OC)cc(O)c2c1=O ; *OC1C(OC(C)=O)OCC(O)C1O]

The said R³ is unsubstituted 6˜10 membered arylC₀₋₂alkyl or unsubstituted 6˜10 membered arylcarbonyl; and

n, m and p are independently 0 or 1.

Preferably,

R¹ and R² are independently unsubstituted or substituted straight or branched C₁₋₃ alkyl; unsubstituted or substituted straight or branched C₁₋₃ alkoxy; unsubstituted or substituted 5˜6 membered cycloalkyl; unsubstituted or substituted 5˜6 membered heterocycloalkyl containing one or more O atoms; or unsubstituted or substituted 6˜8 membered aryl;

Wherein, the substituted C₁₋₃ alkyl and C₁₋₃ alkoxy can be substituted with one or more substituents selected from the group consisting of fluoro, chloro, —OH, unsubstituted phenyl, and unsubstituted phenoxy, and the substituted cycloalkyl, heterocycloalkyl, and aryl can be substituted with one or more substituents selected from the group consisting of fluoro, chloro, —OH, —(CH₂)ₚOR³, methyl, ethyl, propyl, isopropyl, methoxy, ethoxy, propoxy, isopropoxy, unsubstituted phenyl, unsubstituted phenoxy, unsubstituted benzoyl,

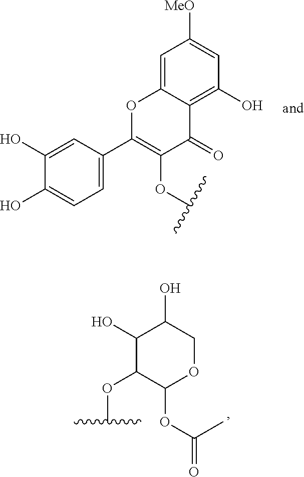 [SMILES: *Oc1c(-c2ccc(O)c(O)c2)oc2cc(OC)cc(O)c2c1=O ; *OC1C(OC(C)=O)OCC(O)C1O]

The said R³ is phenyl or benzoyl; and

n, m and p are independently 0 or 1.

More preferably,

R¹ and R² are independently unsubstituted or substituted cyclohexyl, unsubstituted or substituted tetrahydropyranyl, or unsubstituted or substituted phenyl;

Wherein, the substituted cyclohexyl, tetrahydropyranyl, and phenyl can be substituted with one or more substituents selected from the group consisting of —(CH₂)ₚOR³, methyl, methoxy, isopropoxy,

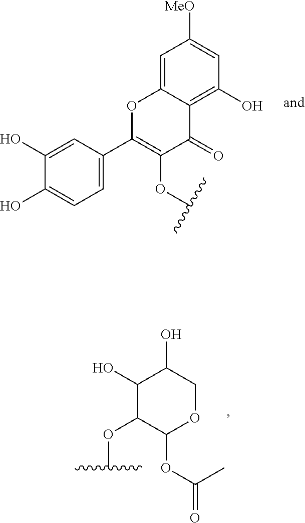 [SMILES: *Oc1c(-c2ccc(O)c(O)c2)oc2cc(OC)cc(O)c2c1=O ; *OC1C(OC(C)=O)OCC(O)C1O]

The said R³ is benzyl or benzoyl; and

n, m and p are independently 0 or 1.

The intermediate compound for the preparation of apiose derivatives represented by formula 4 is produced by performing step 1 of the method for the preparation of apiose derivatives of the present invention, and can be used for the preparation of apiose derivatives through dehydroxylation.

Practical and presently preferred embodiments of the present invention are illustrative as shown in the following Examples.

However, it will be appreciated that those skilled in the art, on consideration of this disclosure, may make modifications and improvements within the spirit and scope of the present invention.

The preparative examples of the present invention are accomplished by the general preparation procedures below.

### GENERAL PREPARATION PROCEDURES OF PREPARATIVE EXAMPLES

### Step 1: General Preparation Procedure of Propargylation

DMF (total conc.: approximately 0.3 M) containing the starting material (1 eq) was added to DMF (dimethylformamide) solution containing NaH (1.2 eq, dispersion in 60% mineral oil) at 0° C. under nitrogen atmosphere. The mixture was stirred at room temperature for 30 minutes. Propargyl bromide (3 eq, 80 wt % in toluene) was added to the mixture above at 0° C. The reaction mixture was stirred at room temperature for 1 hour, followed by quenching with distilled water. The organic residue was extracted by using ethylacetate (Et₂O), dried over sodium sulfate, and then concentrated under reduced pressure. The crude product was isolated by silica gel flash column chromatography.

### Step 2: General Preparation Procedure of Isomerization

t-BuOK (0.4 eq) was added to THF (tetrahydrofuran) (1.0 M) containing the compound obtained in step 1. The reaction mixture was stirred at room temperature until the starting material disappeared. The reaction mixture was filtered with celite and then concentrated under reduced pressure. The crude product was isolated by silica gel flash column chromatography to give allene compound.

### <Preparative Example 1> Preparation of (2R,3R,4S,5R,6R)-3,4-bis(benzyloxy)-2-(benzyloxymethyl)-6-methoxy-5-(propa-1,2-dienyloxy)tetrahydro-2H-pyran

### Step 1: Preparation of (2R,3R,4S,5R,6R)-3,4-bis(benzyloxy)-2-(benzyloxymethyl)-6-methoxy-5-(prop-2-ynyloxy)tetrahydro-2H-pyran

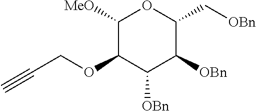 [SMILES: C#CCO[C@H]1[C@H](OC)O[C@H](COCc2ccccc2)[C@@H](OCc2ccccc2)[C@@H]1OCc1ccccc1]

(2R,3R,4S,5R,6R)-3,4-bis(benzyloxy)-2-(benzyloxymethyl)-6-methoxy-5-(prop-2-ynyloxy)tetrahydro-2H-pyran was prepared as a white solid (1.25 g, 2.5 mmol, 75% yield) by the same manner as the general preparation procedure of step 1 except that DMF (11 mL, 0.3 M) containing NaH (188 mg, 3.96 mmol) and methyl 3,4,6-tri-O-benzyl-β-D-glucoside (1.47 g, 3.3 mmol) and propargyl bromide (1.1 mL, 9.9 mmol) were used.

m.p. 68.8-69.8° C. R_{f} 0.47 (Hexane:EtOAc=80:20); [α]²³_{D} −6.69 (c 1.14, CH₂Cl₂); 1H NMR (300 MHz, CDCl₃) δ 7.44-7.17 (m, 15H), 5.03 (d, J=10.5 Hz, 1H), 4.86 (d, J=10.8 Hz, 1H), 4.79 (d, J=10.8 Hz, 1H), 4.64 (d, J=12.0 Hz, 1H), 4.56 (d, J=11.4 Hz, 1H), 4.54-4.49 (m, 3H), 4.42 (dd, J=10.8, 2.1 Hz, 1H), 4.27 (d, J=7.5 Hz, 1H), 3.76 (dd, J=10.8, 2.1 Hz, 1H), 3.70 (dd, J=10.8, 4.5 Hz, 1H), 3.65-3.60 (m, 2H), 3.57 (s, 3H), 3.48-3.41 (m, 2H), 2.47 (t, J=2.4 Hz, 1H); ¹³C NMR (75 MHz, CDCl₃) δ 138.7, 138.33, 138.30, 128.6, 128.54, 128.46, 128.1, 128.0, 127.9, 127.8, 104.5, 84.4, 81.8, 80.3, 77.8, 75.9, 75.3, 75.0, 74.5, 73.7, 69.0, 59.6, 57.2; IR (NaCl) ν 3288, 3088, 3064, 3030, 2911, 2866, 2118, 1606, 1497, 1454, 1149 cm⁻¹.

### Step 2: Preparation of (2R,3R,4S,5R,6R)-3,4-bis(benzyloxy)-2-(benzyloxymethyl)-6-methoxy-5-(propa-1,2-dienyloxy)tetrahydro-2H-pyran

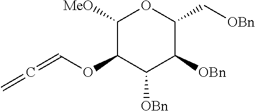 [SMILES: C=C=CO[C@H]1[C@H](OC)O[C@H](COCc2ccccc2)[C@@H](OCc2ccccc2)[C@@H]1OCc1ccccc1]

(2R,3R,4S,5R,6R)-3,4-bis(benzyloxy)-2-(benzyloxymethyl)-6-methoxy-5-(propa-1,2-dienyloxy)tetrahydro-2H-pyran was prepared as a white solid (348 mg, 0.69 mmol, 70% yield) by the same manner as the general preparation procedure of step 2 except that THF (1.0 mL, 1.0 M) containing the compound obtained in step 1 (506 mg, 1 mmol) and t-BuOK (70 mg, 0.6 mmol) were used.

m.p. 40.0-42.9° C. R_{f} 0.47 (Hexane:EtOAc=90:10); [α]²⁵_{D} −20.31 (c 0.94, CH₂Cl₂); 1H NMR (300 MHz, CDCl₃) 7.35-7.16 (m, 15H), 6.80 (t, J=6.0 Hz, 1H), 5.49 (dd, J=6.0, 1.5 Hz, 2H), 4.91 (d, J=10.7 Hz, 1H), 4.83 (d, J=10.8 Hz, 1H), 4.75 (d, J=10.7 Hz, 1H), 4.63 (d, J=12.2 Hz, 1H), 4.55 (d, J=12.2 Hz, 1H), 4.53 (d, J=10.8 Hz, 1H), 4.32 (dd, J=4.9, 2.5 Hz, 1H), 3.76 (dd, J=10.8, 2.1 Hz, 1H) 3.73-3.68 (m, 3H), 3.65-3.60 (m, 1H), 3.56 (s, 1H), 3.48 (ddd, J=9.4, 4.4, 2.1 Hz, 1H); ¹³C NMR (75 MHz, CDCl₃) δ 201.3, 138.5, 138.31, 138.26, 128.61, 128.56, 128.4, 128.1, 127.98, 127.93, 127.8, 122.9, 103.3, 90.9, 84.4, 81.9, 77.8, 75.6, 75.3, 75.1, 73.7, 69.0, 57.4; IR (NaCl) ν 3388, 3069, 3063, 3031, 3006, 2915, 2867, 1959, 1877, 1811, 1733, 1060 cm⁻¹; HRMS (FAB) calcd for C₃₁H₃₅O₆ (MH⁺) 503.2434, found 503.2430.

### <Preparative Example 2> Preparation of (2S,3R,4S,5R)-2,3,5-tris(benzyloxy)-4-(propa-1,2-dienyloxy)tetrahydro-2H-pyran

### Step 1: Preparation of (2S,3R,4S,5R)-2,3,5-tris(benzyloxy)-4-(prop-2-ynyloxy)tetrahydro-2H-pyran

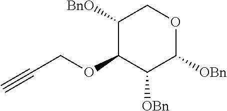 [SMILES: C#CCO[C@@H]1[C@@H](OCc2ccccc2)[C@@H](OCc2ccccc2)OC[C@H]1OCc1ccccc1]

(2S,3R,4S,5R)-2,3,5-tris(benzyloxy)-4-(prop-2-ynyloxy)tetrahydro-2H-pyran was prepared as a colorless oil (3.77 g, 8.22 mmol, 93% yield) by the same manner as the general preparation procedure of step 1 except that DMF (11 mL, 0.3 M) containing NaH (480 mg, 10.6 mmol, 60% dispersion in mineral oil) and 1,2,4-tri-O-benzyl-D-xylopyranose (3.71 g, 8.83 mmol) and propargyl bromide (2.9 mL, 26.5 mmol, 80 wt % in toluene) were used.

R_{f} 0.52 (Hexane:EtOAc=80:20); [α]²⁵_{D} +75.89 (c 0.49, CH₂Cl₂); 1H NMR (300 MHz, CDCl₃) δ 7.37-7.29 (m, 15H), 4.82 (d, J=11.4 Hz, 1H), 4.76-4.47 (m, 8H), 3.85 (dd, J=9.3, 8.1 Hz, 1H), 3.62-3.50 (m, 3H), 3.41 (dd, J=11.4, 9.3 Hz, 2H), 2.48 (t, J=2.4 Hz, 1H); ¹³C NMR (75 MHz, CDCl₃) δ 138.5, 138.4, 137.3, 128.64, 128.61, 128.58, 128.5, 128.08, 128.01, 127.95, 95.4, 81.6, 80.7, 79.7, 77.7, 74.2, 73.9, 73.3, 68.9, 60.8, 60.3; IR (NaCl) ν 3289, 3089, 3064, 3031, 2934, 2884, 2120, 1955, 1725, 1603, 1586, 1497, 1454, 1365, 1332, 1271 cm⁻¹.

### Step 2: Preparation of (2S,3R,4S,5R)-2,3,5-tris(benzyloxy)-4-(propa-1,2-dienyloxy)tetrahydro-2H-pyran

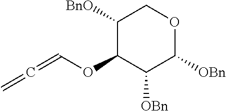 [SMILES: C=C=CO[C@@H]1[C@@H](OCc2ccccc2)[C@@H](OCc2ccccc2)OC[C@H]1OCc1ccccc1]

(2S,3R,4S,5R)-2,3,5-tris(benzyloxy)-4-(propa-1,2-dienyloxy)tetrahydro-2H-pyran was prepared as a white solid (284.5 mg, 0.62 mmol, 94% yield) by the same manner as the general preparation procedure of step 2 except that THF (0.65 mL, 1.0 M) containing the compound obtained in step 1 (316 mg, 0.67 mmol) and t-BuOK (26 mg, 0.2 mmol) were used.

m.p. 40.0-42.9° C. R_{f} 0.74 (Hexane:EtOAc=80:20); [α]²²_{D} +74.22 (c 0.98, CHCl₃); ¹H NMR (500 MHz, CDCl₃) δ 7.37-7.22 (m, 15H), 6.86 (t, J=5.8 Hz, 1H), 5.40 (dd, J=3.5, 2.5 Hz, 2H), 4.76 (d, J=11.5 Hz, 1H), 4.71-4.46 (m, 6H), 4.12 (t, J=8.8 Hz, 1H), 3.60-3.54 (m, 3H), 3.41 (dd, J=6.0, 3.5 Hz, 1H); ¹³C NMR (125 MHz, CDCl₃) δ 201.8, 138.4, 138.4, 137.3, 128.6, 128.6, 128.5, 128.3, 128.1, 128.0, 128.0, 127.9, 123.6, 95.8, 89.9, 82.7, 78.7, 77.3, 73.8, 73.4, 68.9, 60.2; IR (NaCl) ν 3088, 3064, 2935, 2884, 1960, 1497, cm⁻¹; HRMS (FAB) calcd for C₂₉H₃₁O₅ (MH⁺) 459.2171, found 459.2167.

### <Preparative Example 3> Preparation of (2R,3R,4S,5R,6R)-2,3,4,5-tetrakis(benzyloxy)-6-((propa-1,2-dienyloxy)methyl)tetrahydro-2H-pyran

### Step 1: Preparation of (2R,3R,4S,5R,6R)-2,3,4,5-tetrakis(benzyloxy)-6-((prop-2-ynyloxy)methyl)tetrahydro-2H-pyran

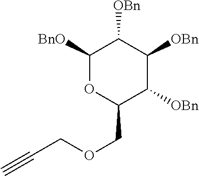 [SMILES: C#CCOC[C@H]1O[C@@H](OCc2ccccc2)[C@H](OCc2ccccc2)[C@@H](OCc2ccccc2)[C@@H]1OCc1ccccc1]

(2R,3R,4S,5R,6R)-2,3,4,5-tetrakis(benzyloxy)-6-((prop-2-ynyloxy)methyl)tetrahydro-2H-pyran was prepared as a white solid (3.45 g, 5.96 mmol, 98.7% yield) by the same manner as the general preparation procedure of step 1 except that DMF (11 mL, 0.3 M) containing NaH (320 mg, 6.15 mmol, 60% dispersion in mineral oil) and 1,2,4-tri-O-benzyl-D-xylopyranose (3.77 g, 5.12 mmol) and propargyl bromide (1.37 mL, 15.4 mmol, 80 wt % in toluene) were used.

m.p. 88.9-90.4° C. R_{f} 0.51 (Hexane:EtOAc=80:20); [α]²¹_{D} −14.23 (c 0.89, CH₂Cl₂); 1H NMR (300 MHz, CDCl₃) 7.42-7.31 (m, 20H), 5.02-4.67 (m, 8H), 4.54 (d, J=7.5 Hz, 1H), 4.24 (ddd, J=15.9, 10.1, 2.4 Hz, 2H), 3.90-3.80 (m, 2H), 3.70-3.63 (m, 2H), 3.58-3.50 (m, 2H), 2.41 (t, J=2.1 Hz, 1H); ¹³C NMR (75 MHz, CDCl₃) 138.8, 138.5, 138.4, 137.6, 128.59, 128.57, 128.5, 128.4, 128.2, 128.1, 128.0, 127.96, 127.94, 127.84, 127.79, 102.9, 84.8, 82.4, 79.9, 77.8, 75.9, 75.2, 75.1, 75.0, 74.8, 71.4, 68.5, 58.9; IR (NaCl) ν 3287, 3088, 3063, 3031, 2870, 1952, 1606, 1497, 1454, 1399, 1360 cm⁻¹; HRMS (FAB) calcd for C₃₇H₃₉O₆ (MH⁺) 579.2747, found 579.2751.

### Step 2: Preparation of (2R,3R,4S,5R,6R)-2,3,4,5-tetrakis(benzyloxy)-6-((propa-1,2-dienyloxy)methyl)tetrahydro-2H-pyran

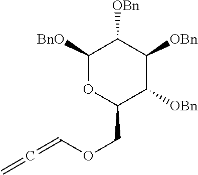 [SMILES: C=C=COC[C@H]1O[C@@H](OCc2ccccc2)[C@H](OCc2ccccc2)[C@@H](OCc2ccccc2)[C@@H]1OCc1ccccc1]

(2R,3R,4S,5R,6R)-2,3,4,5-tetrakis(benzyloxy)-6-((propa-1,2-dienyloxy)methyl)tetrahydro-2H-pyran was prepared as a white solid (490.5 mg, 0.85 mmol, 92% yield) by the same manner as the general preparation procedure of step 2 except that THF (0.9 mL, 1.0 M) containing the compound obtained in step 1 (536 mg, 0.93 mmol) and t-BuOK (26 mg, 0.2 mmol) were used.

m.p. 78.7-79.8° C. R_{f} 0.35 (Hexane:EtOAc=80:20); [α]²²_{D} −24.25 (c 0.94, CH₂Cl₂); 1H NMR (300 MHz, CDCl₃) 7.44-7.32 (m, 20H), 6.89 (t, J=5.94 Hz, 1H), 5.52 (dd, J=5.9, 4.0 Hz, 2H), 5.06-4.85 (m, 5H), 4.79 (d, J=11.7 Hz, 1H), 4.72 (d, J=12.0 Hz, 1H), 4.65 (d, J=10.8 Hz, 1H), 4.60 (d, J=7.65 Hz, 1H), 3.89-3.86 (m, 2H), 3.74-3.69 (m, 2H), 3.63-3.54 (m, 2H); ¹³C NMR (75 MHz, CDCl₃) δ 201.1, 138.7, 138.5, 138.2, 137.5, 128.61, 128.55, 128.54, 123.50, 128.34, 128.28, 128.1, 128.0, 127.9, 127.8, 127.78, 122.0, 102.7, 91.4, 84.8, 82.4, 75.69, 75.3, 75.1, 73.9, 71.3, 67.3; IR (NaCl) ν 3089, 3064, 3030, 2924, 2876, 1950, 1726, 1606, 1497, 1453, 1445, 1406, 1352 cm⁻¹; HRMS (FAB) calcd for C₃₇H₃₉O₆ (MH⁺) 579.2747, found 579.2744.

### <Preparative Example 4> Preparation of (2R,3R,4S,5R,6R)-2-(benzoyloxymethyl)-6-(2-(hydroxymethyl)allyloxy)tetrahydro-2H-pyran-3,4,5-triyl tribenzoate

### Step 1: Preparation of (2R,3R,4S,5R,6R)-2-(benzoyloxymethyl)-6-(2-((triisopropylsilyloxy)methyl)allyloxy)tetrahydro-2H-pyran-3,4,5-triyl tribenzoate

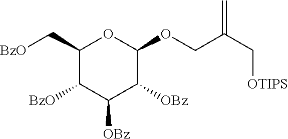

Zinc bromide and powder type molecular sieve 4 A were vacuum-dried at 110° C. for 1 hour. A mixture of 2,3,4,6-tetra-o-benzoyl-alpha-d-glucoglucopyranosyl bromide, 2-((triisopropylsilyloxy)methyl)prop-2-en-1-ol (444 mg, 1.82 mmol), ZnBr₂ (440 mg, 1.82 mmol), and molecular sieve 4 A (1.00 g) was added to CH₂Cl₂ (23 mL). The prepared mixture suspension was stirred until the starting material glucopyranosyl bromide disappeared. The reaction mixture was quenched by dilution with EtOAc (50 mL) and the aqueous solution (20 mL) containing NaHCO₃ (160 mg) and Na₂S₂O₃ (240 mg). The mixture was stirred for 10 minutes, filtered with celite, and washed with EtOAc (50 mL). The organic layer was washed with NaCl solution (20 mL). The combined aqueous solution layer was extracted with EtOAc (2×50 mL). The combined organic layer was dried over sodium sulfate. The solvent was eliminated and the obtained residue proceeded to silica gel flash column chromatography (eluted with Hexane/Et₂O=70:30) to give (2R,3R,4S,5R,6R)-2-(benzoyloxymethyl)-6-(2-((triisopropylsilyloxy)methyl)allyloxy)tetrahydro-2H-pyran-3,4,5-triyl tribenzoate as a colorless oil (803 mg, 0.98 mmol, 65% yield).

R_{f} 0.53 (Hexane:EtOAc=80:20); [α]³⁰_{D} +7.8 (c 0.5, CH₂Cl₂); 1H NMR (300 MHz, CDCl₃) δ 8.05-8.03 (m, 2H), 7.97-7.94 (m, 2H), 7.92-7.89 (m, 2H), 7.84-7.81 (m, 2H), 7.55-7.24 (m, 12H), 5.92 (t, J=9.63 Hz, 1H), 5.69 (t, J=9.6 Hz, 1H), 5.58 (dd J=7.9, 9.8 Hz, 1H), 5.25 (d, J=1.6 Hz, 1H), 5.07 (s, 1H), 4.90 (d, J=7.9, 1H), 4.65 (dd, J=12.1, 3.2 Hz, 1H), 4.52 (dd, J=12.2, 5.4 Hz, 1H), 4.39 (d, J=12.4 Hz, 1H), 4.22 (d, J=12.5 Hz, 1H), 4.19-4.13 (m, 1H), 4.09 (s, 2H), 0.95 (s, 21H); ¹³C NMR (75 MHz, CDCl₃) δ 166.3, 166.0, 165.4, 165.2, 143.9, 133.6, 133.4, 133.3, 130.0, 129.95, 129.93, 129.91, 129.8, 129.4, 129.0, 128.59, 128.56, 128.5, 113.4, 99.5, 73.2, 72.4, 72.0, 70.0, 69.9, 63.8, 63.4, 18.1, 12.0; IR (NaCl) ν 3064, 2943, 2865, 1733, 1602, 1585, 1492, 1452, 1368, 1315, 1266, 1178, 1094, 1069, 1027, 918, 883 cm⁻¹.

### Step 2: Preparation of (2R,3R,4S,5R,6R)-2-(benzoyloxymethyl)-6-(2-(hydroxymethyl)allyloxy)tetrahydro-2H-pyran-3,4,5-triyl tribenzoate

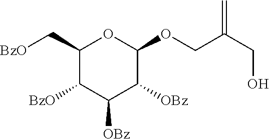

THF (3.33 mL, 0.3 M) containing the compound (803.1 mg, 0.98 mmol) obtained in step 1 was treated with 1.2 mmol of TBAF (THF of 1 M aqueous solution). The reaction solution was stirred at 0° C. for 1-2 hours. The solution was diluted with distilled water, followed by extraction with EtOAc (2×20 mL). The combined organic layer was dried over sodium sulfate and the solvent was concentrated under reduced pressure. The residue proceeded to silica gel column chromatography (eluted with Hexane/Et₂O=50:50) to give (2R,3R,4S,5R,6R)-2-(benzoyloxymethyl)-6-(2-(hydroxymethyl)allyloxy)tetrahydro-2H-pyran-3,4,5-triyl tribenzoate as a white solid (554 mg, 0.83 mmol, 85% yield).

m.p. 49-51° C. R_{f} 0.53 (Hexane:EtOAc=50:50); [α]³⁰_{D} +16.7 (c 0.7, CH₂Cl₂); 1H NMR (300 MHz, CDCl₃) δ 8.06-8.03 (m, 2H), 7.97-7.95 (m, 2H), 7.92-7.89 (m, 2H), 7.84-7.81 (m, 2H), 7.56-7.25 (m, 12H), 5.92 (t, J=9.7 Hz, 1H), 5.68 (t, J=9.7 Hz, 1H), 5.55 (dd J=9.7, 7.9 Hz, 1H), 5.13 (s, 1H), 5.07 (s, 1H), 4.90 (d, J=7.8, 1H), 4.69 (dd, J=12.2, 3.1 Hz, 1H), 4.49 (dd, J=12.2, 5.3 Hz, 1H), 4.43 (d, J=12.3 Hz, 1H), 4.23 (d, J=12.4 Hz, 1H), 4.16 (ddd, J=9.7, 5.1 Hz, 3.2 Hz, 1H), 4.04 (d, J=3.1 Hz, 2H), 1.77 (br, 1H); ¹³C NMR (75 MHz, CDCl₃) δ 166.4, 166.0, 165.38, 165.35, 144.4, 133.7, 133.6, 133.5, 133.4, 130.0, 129.96, 129.7, 129.3, 128.9, 128.6 128.5, 114.4, 100.3, 73.0, 72.6, 72.1, 70.8, 69.8, 63.8, 63.1; IR (NaCl) ν 3203, 3167, 3090, 3064, 3034, 1371, 1351, 1316, 1269, 1178, 1159, 1107 cm⁻¹; HRMS (ESI) calcd for C₃₈H₃₄O₁₁Na⁺ (M+Na⁺) 689.1993, found 689.1994.

The examples of the present invention are accomplished by the general preparation procedures below.

### GENERAL PREPARATION PROCEDURES OF EXAMPLES

### Step 1: General Preparation Procedure of Pd-Catalyzed Hydroalkoxylation

Toluene containing the allene starting material (1 eq) and the alcohol starting material (1.5 eq) and toluene containing triethylamine (0.1-1.5 eq) were added to the solution containing Pd₂(dba)₃ (approximately 2.5 mol %) and (R,R)-L1 or (S,S)-L1 (3 mol %) under nitrogen atmosphere. The reaction mixture was stirred at 40° C. until the starting material disappeared. The crude product was purified by silica gel column chromatography to give the target compound.

### Step: General Preparation Procedure of Ring Closing Metathesis

1^{st} Grubbs' catalyst (3-15 mol %) was added to the acyclic O,O-acetal (1 eq) obtained in step 1 above and dissolved in CH₂Cl₂ (0.05 M). The reaction mixture was stirred until the starting material disappeared. The solvent was concentrated under reduced pressure. The crude product was purified by silica gel column chromatography to give the target compound.

### Step 3: General Preparation Procedure of Dihydroxylation of Cyclic O,O-Acetal

4-Methylmorpholine N-oxide (2 eq) and OsO₄ solution (4 wt % in H₂O, approximately 0.003 eq) were added to acetone/THF (1:1 (v:v), 1.0 M) containing the cyclic O,O-acetal obtained in step 2 above, to which distilled water (total volume=1.4 M) was added at 0° C. The reaction mixture was stirred until the starting material disappeared. The reaction mixture was diluted with CH₂Cl₂ and washed with 10% Na₂SO₃ aqueous solution and saturated NH₄Cl. The combined aqueous solution layer was extracted with CH₂Cl₂. The combined organic layer was dried over anhydrous sodium sulfate and concentrated under reduced pressure. The crude product was isolated by flash column chromatography to give the target compound.

### <Example 1> Preparation of (2S,3R,4R)-4-(benzyloxymethyl)-2-(cyclohexyloxy)tetrahydrofuran-3,4-diol

### Step 1: Preparation of (S)-((2-((1-(cyclohexyloxy)allyloxy)methyl)allyloxy)methyl)benzene

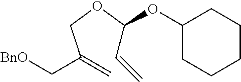 [SMILES: C=C[C@@H](OCC(=C)COCc1ccccc1)OC1CCCCC1]

(S)-((2-((1-(cyclohexyloxy)allyloxy)methyl)allyloxy)methyl)benzene was prepared as a colorless oil (142.0 mg, 0.45 mmol, 96% yield) by the same general procedure of step 1 by using (propa-1, 2-dienyloxy)cyclohexane (120 mg, 0.87 mmol), 2-(benzyloxymethyl)prop-2-en-1-ol (83 mg, 0.47 mmol), Pd₂(dba)₃ (10.0 mg, 11.0 μmol), (R, R)-L1 (15.0 mg, 21.1 μmol), and triethylamine (6.0 μL, 0.47 mmol).

R_{f} 0.47 (Hexane:EtOAc=90:10); [α]²⁹_{D} −17.5 (c 0.76, CH₂Cl₂); 1H NMR (300 MHz, CDCl₃) δ 7.37-7.28 (m, 5H), 5.88 (ddd, J=17.3, 10.5, 4.9 Hz, 1H), 5.41 (dt, J=17.1, 1.3 Hz, 1H) 5.30-5.24 (m, 3H), 5.04 (dt, J=1.0 Hz, 18.3 Hz, 1H), 4.53 (s, 2H), 4.15 (d, J=12.8 Hz, 1H), 4.07 (d, J=12.6 Hz, 1H), 4.07 (s, 2H), 3.63-3.54 (m, 1H), 1.9-1.73 (m, 4H), 1.14-1.19 (m, 6H); ¹³C NMR (125 MHz, CDCl₃) δ 143.0, 138.5, 136.1, 128.6, 127.9, 127.8, 118.2, 114.2, 99.9, 77.7, 74.6, 72.3 71.3, 65.7, 33.5, 32.8, 25.9, 24.5, 24.4; IR (NaCl) ν 3030, 2933, 2856, 1657, 1496, 1453, 1407, 1360, 1260, 1094 cm⁻¹; HRMS (FAB) calcd for C₂₀H₂₉O₃ (MH⁺) 317.2117, found 317.2113.

### Step 2: Preparation of (S)-4-(benzyloxymethyl)-2-(cyclohexyloxy)-2,5-dihydrofuran

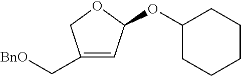 [SMILES: C1=C(COCc2ccccc2)CO[C@H]1OC1CCCCC1]

(S)-4-(benzyloxymethyl)-2-(cyclohexyloxy)-2, 5-dihydrofuran was prepared as a colorless oil (36.6 mg, 0.116 mmol, 86% yield) by the same general procedure of step 2 above by using the compound obtained in step (42.2 mg, 0.134 mmol) and CH₂Cl₂ (1.3 mL) containing 1^{st} Grubbs' catalyst (3 mg, 0.005 mmol).

R_{f} 0.33 (Hexane:EtOAc=90:10); [α]²⁹_{D} −25.5 (c 0.90, CH₂Cl₂); 1H NMR (300 MHz, CDCl₃) δ 7.36-7.29 (m, 5H), 5.95 (d, J=4.3 Hz, 1H), 5.71 (d, J=1.2 Hz, 1H), 4.74-4.68 (m, 1H), 4.57-4.52 (m, 3H), 4.19-4.18 (m, 2H), 3.63-3.54 (m, 1H), 2.00-1.92 (m, 2H), 1.76-1.69 (m, 2H), 1.57-1.14 (m, 6H); ¹³C NMR (125 MHz, CDCl₃) δ 143.4, 137.9, 128.5, 127.9, 127.8, 122.8, 107.8, 76.4, 74.0, 72.7, 65.6, 34.1, 32.8, 25.7, 24.54, 24.51; IR (NaCl) ν 3064, 3031, 2932, 2856, 1674, 1497, 1453, 1357, 1317, 1260, 1246, 1199, 1156, cm⁻¹; HRMS (FAB) calcd for C₁₈H₂₅O₃ (MH⁺), 289.1804 found 289.1802.

### Step 3: Preparation of (2S,3R,4R)-4-(benzyloxymethyl)-2-(cyclohexyloxy)tetrahydrofuran-3, 4-diol

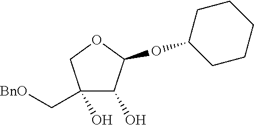

A target compound was prepared as a colorless oil (67.7 mg, 0.21 mmol, 99.9% yield) by the same general procedure of step 3 above by using acetone/THF (1:1(v:v), 0.20 mL) containing the compound obtained in step 2 (59.5 mg, 0.21 mmol), 4-methylmorpholine N-oxide (49.0 mg, 0.42 mmol), OsO₄ solution (4 wt % in H2O, 40.0 μL, 5.7 μmol), and distilled water (0.03 mL, total volume: 0.07 mL, 3.0 M).

R_{f} 0.55 (Hexane:EtOAc=50:50); [α]²⁹_{D} −73.6 (c 0.49, CH₂Cl₂); 1H NMR (300 MHz, CDCl₃) δ 7.36-7.29 (m, 5H), 5.95 (d, J=4.3 Hz, 1H), 5.71 (d, J=1.2 Hz, 1H), 4.74-4.68 (m, 1H), 4.57-4.52 (m, 3H), 4.19-4.18 (m, 2H), 3.63-3.54 (m, 1H), 2.00-1.92 (m, 2H), 1.76-1.69 (m, 2H), 1.57-1.14 (m, 6H); ¹³C NMR (125 MHz, CDCl₃) 5137.6, 128.8, 128.2, 128.0, 106.8, 78.9, 78.2, 75.6, 74.0, 73.8, 73.4, 33.7, 31.8, 25.8, 24.3, 24.1; IR (NaCl) ν 3403, 2932, 2856, 1497, 1453, 1363, 1092, 1005, 947 cm⁻¹; HRMS (FAB) calcd for C₁₈H₂₇O₅ (MH⁺), 323.1858 found 323.1861.

### <Example 2> Preparation of (2S,3R,4R)-4-(benzyloxymethyl)-2-((2R,3R,4S,5R,6R)-4, 5-bis(benzyloxy)-6-(benzyloxymethyl)-2-methoxytetrahydro-2H-pyran-3-yloxy)tetrahydrofuran-3, 4-diol

### Step 1: Preparation of (2R,3R,4S,5R,6R)-3, 4-bis(benzyloxy)-2-(benzyloxymethyl)-5-((S)-1-(2-(benzyloxymethyl)allyloxy)allyloxy)-6-methoxytetrahydro-2H-pyran

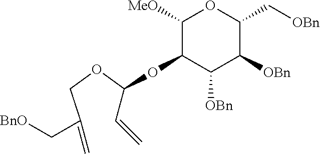 [SMILES: C=C[C@@H](OCC(=C)COCc1ccccc1)O[C@H]1[C@H](OC)O[C@H](COCc2ccccc2)[C@@H](OCc2ccccc2)[C@@H]1OCc1ccccc1]

(2R,3R,4S,5R,6R)-3, 4-bis(benzyloxy)-2-(benzyloxymethyl)-5-((S)-1-(2-(benzyloxymethyl)allyloxy)allyloxy)-6-methoxytetrahydro-2H-pyran was prepared as a colorless oil (115.3 mg, 0.16 mmol, 82% yield) by the same general procedure of step 1 by using the compound obtained in Preparative Example 1 (104 mg, 0.20 mmol), 2-(benzyloxymethyl)prop-2-en-1-ol (56 mg, 0.31 mmol), Pd₂(dba)₃ (5.8 mg, 6.3 μmol), (R, R)-L1 (7.6 mg, 0.011 mmol), and triethylamine (2.4 μL, 0.018 mmol).

R_{f} 0.37 (Hexane:EtOAc=80:20); [α]²⁵_{D} −24.6 (c 0.76, CH₂Cl₂); 1H NMR (500 MHz, CDCl₃) δ 7.38-7.11 (m, 20H), 5.82 (ddd, J=17.4, 10.5, 6.2 Hz, 1H), 5.32 (d, J=17.4 Hz, 1H), 5.25-5.21 (m, 4H), 4.90 (d, J=11.0 Hz, 1H), 4.79 (d, J=11.0 Hz, 1H), 4.78 (d, J=12.2 Hz, 1H), 4.62 (d, J=12.2 Hz, 1H), 4.55 (d, J=12.2 Hz, 1H), 4.52-4.50 (m, 3H), 4.29 (d, J=7.7 Hz, 1H), 4.28 (d, J=12.8 Hz, 1H), 4.17 (d, J=12.8 Hz, 1H), 4.07 (s, 2H), 3.74 (dd, J=10.9, 2.0 Hz, 1H), 3.68 (dd, J=10.8, 4.7 Hz, 1H), 3.63-3.56 (m, 2H), 3.51 (s, 3H), 3.53-3.47 (m, 2H); ¹³C NMR (125 MHz, CDCl₃) δ 143.0, 138.7, 138.6, 138.4, 138.3, 135.4, 128.59, 128.57, 128.53, 128.1, 127.99, 127.96, 127.94, 127.88, 127.82, 127.76, 118.9, 114.0, 104.2, 103.8, 84.8, 78.2, 77.8, 75.9, 75.2, 75.1, 73.7, 72.3, 71.3, 69.1, 67.4, 57.0; IR (NaCl) ν 3088, 3064, 3030, 2922, 2860, 1740, 1658, 1606, 1497, 1454, 1361, 1309, 1278, 1215, 1095, 1058 cm⁻¹; HRMS (ESI) calcd for C₄₂H₄₈O₈Na⁺ (M+Na⁺) 703.3241, found 703.3240.

### Step 2: Preparation of (2R,3R,4S,5R,6R)-3,4-bis(benzyloxy)-2-(benzyloxymethyl)-5-((S)-4-(benzyloxymethyl)-2,5-dihydrofuran-2-yloxy)-6-methoxytetrahydro-2H-pyran

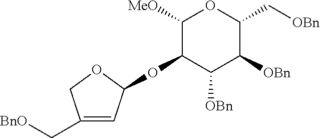 [SMILES: CO[C@@H]1O[C@H](COCc2ccccc2)[C@@H](OCc2ccccc2)[C@H](OCc2ccccc2)[C@H]1O[C@H]1C=C(COCc2ccccc2)CO1]

(2R,3R,4S,5R,6R)-3, 4-bis(benzyloxy)-2-(benzyloxymethyl)-5-((S)-4-(benzyloxymethyl)-2, 5-dihydrofuran-2-yloxy)-6-methoxytetrahydro-2H-pyran was prepared as a colorless oil (63.4 mg, 0.097 mmol, 77% yield) by the same general procedure of step 2 above by using the compound obtained in step 1 (87 mg, 0.13 mmol) and CH₂Cl₂ (1.3 mL) containing 1^{st} Grubbs' catalyst (11 mg, 0.013 mmol).

R_{f} 0.50 (Hexane:EtOAc=80:20); [α]²⁶_{D} +4.39 (c 0.24, CH₂Cl₂); 1H NMR (500 MHz, CDCl₃) δ 7.34-7.14 (m, 20H), 6.07 (d, J=6.8 Hz, 1H), 5.68 (s, 1H), 4.88 (d, J=11 Hz, 1H), 4.83-4.78 (m, 2H), 4.74-4.72 (m, 1H), 4.63 (d, J=12 Hz, 1H), 4.56 (d, J=12 Hz, 1H), 4.55 (d, J=11 Hz, 1H), 4.53-4.47 (m, 3H), 4.28 (d, J=7 Hz, 1H), 4.18 (s, 2H), 3.76 (dd, J=11.0, 2.0 Hz, 1H), 3.69 (dd, J=11.0, 4.5 Hz, 1H), 3.62-3.60 (m, 3H), 3.47-3.46 (m, 1H); ¹³C NMR (125 MHz, CDCl₃) δ 143.7, 139.1, 138.5, 138.4, 138.0, 128.67, 128.63, 128.60, 128.5, 128.27, 128.26, 128.01, 127.96, 127.91, 127.8, 127.7, 122.8, 110.5, 104.5, 84.0, 80.3, 77.9, 75.5, 75.3, 75.2, 74.3, 73.7, 72.8, 69.3, 65.6, 57.3; IR (NaCl) ν 2924, 2865, 2844, 1497, 1454, 1359, cm⁻¹; HRMS (ESI) calcd for C₄₀H₄₄O₈Na⁺ (M+Na⁺) 675.2928, found 675.2926.

### Step 3: Preparation of (2S,3R,4R)-4-(benzyloxymethyl)-2-((2R,3R,4S,5R,6R)-4,5-bis(benzyloxy)-6-(benzyloxymethyl)-2-methoxytetrahydro-2H-pyran-3-yloxy)tetrahydrofuran-3,4-diol

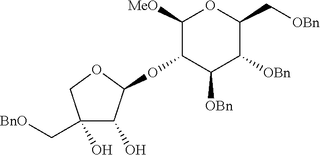 [SMILES: CO[C@H]1O[C@@H](COCc2ccccc2)[C@H](OCc2ccccc2)[C@@H](OCc2ccccc2)[C@@H]1O[C@@H]1OC[C@](O)(COCc2ccccc2)[C@H]1O]

A target compound was prepared as a yellow syrup (36.5 mg, 0.057 mmol, 58% yield) by the same general procedure of step 3 above by using acetone/THF (1:1(v:v), 0.8 mL) containing the compound obtained in step 2 (58.0 mg, 0.089 mmol), 4-methylmorpholine N-oxide (16 mg, 0.18 mmol), OsO₄ solution (4 wt % in H2O, 10.0 μL, 1.5 μmol), and distilled water (0.07 mL, total volume: 0.08 mL, 1.2 M).

R_{f} 0.40 (Hexane:EtOAc=50:50); [α]²⁹_{D} −44.33 (c 0.23, CH₂Cl₂); ¹H NMR (300 MHz, CDCl₃) δ 7.38-7.17 (m, 20H), 5.34 (s, 1H), 4.84 (s, 2H), 4.80 (d, J=10.8 Hz, 1H), 4.63 (d, J=12.2 Hz, 1H), 4.58-4.51 (m, 4H), 4.10 (d, J=4.9 Hz, 1H), 3.93 (d, J=10.0 Hz, 1H), 3.88-3.84 (m, 2H), 3.75 (dd, J=10.9, 1.9 Hz, 1H) 3.69 (dd, J=10.9, 4.6 Hz, 1H) 3.60-3.54 (m, 4H), 3.50 (d, J=9.5 Hz 1H), 3.44-3.40 (m, 4H), 3.06 (br, 1H), 2.83 (br, 1H); ¹³C NMR (75 MHz, CDCl₃) δ 138.3, 138.2, 137.7, 128.71, 128.68, 128.6, 128.5, 128.3, 128.2, 128.0, 127.9, 127.8, 109.1, 103.1, 85.2, 79.1, 78.4, 78.0, 75.8, 75.3, 75.1, 74.4, 73.72, 73.67, 73.1, 69.0, 56.8; IR (NaCl) ν 3445, 3063, 3030, 2926, 2861, 1733, 1497, 1454, 1362, 1261, 1215, 1073, 1028, 821, 737 cm⁻¹; HRMS (ESI) calcd for C₄₀H₄₆O₁₀Na⁺ (M+Na⁺) 709.2983, found 709.2982.

### <Example 3> Preparation of (2R,3R,4R)-4-(benzyloxymethyl)-2-((2R,3R,4S,5R,6R)-4, 5-bis(benzyloxy)-6-(benzyloxymethyl)-2-methoxytetrahydro-2H-pyran-3-yloxy)tetrahydrofuran-3, 4-diol

### Step 1: Preparation of (2R,3R,4S,5R,6R)-3, 4-bis(benzyloxy)-2-(benzyloxymethyl)-5-((R)-1-(2-(benzyloxymethyl)allyloxy)allyloxy)-6-methoxytetrahydro-2H-pyran

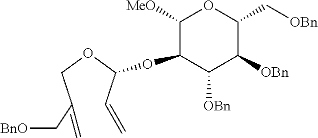 [SMILES: C=C[C@H](OCC(=C)COCc1ccccc1)O[C@H]1[C@H](OC)O[C@H](COCc2ccccc2)[C@@H](OCc2ccccc2)[C@@H]1OCc1ccccc1]

(2R,3R,4S,5R,6R)-3, 4-bis(benzyloxy)-2-(benzyloxymethyl)-5-((R)-1-(2-(benzyloxymethyl)allyloxy)allyloxy)-6-methoxytetrahydro-2H-pyran was prepared as a colorless oil (118 mg, 0.17 mmol, 87% yield) by the same general procedure of step 1 by using the compound obtained in Preparative Example 1 (101 mg, 0.20 mmol), 2-(benzyloxymethyl)prop-2-en-1-ol (54.4 mg, 0.30 mmol), Pd₂ (dba)₃ (5.7 mg, 5.0 μmol), (S, S)-L1 (7.4 mg, 0.01 mmol), and triethylamine (2.0 μL, 0.02 mmol).

R_{f} 0.33 (Hexane:EtOAc=90:10); [α]²⁶_{D} −8.5 (c 0.24, CH₂Cl₂); 1H NMR (500 MHz, CDCl₃) δ 7.37-7.11 (m, 20H), 5.86 (ddd, J=17.3, 10.5, 5.0 Hz, 1H), 5.39 (d, J=17.3 Hz, 1H), 5.31 (d, J=5.0 Hz, 1H), 5.30 (s, 1H), 5.26 (dd, J=10.0, 1.0 Hz, 1H), 5.14 (s, 2H), 5.07 (d, J=11.0 Hz, 1H), 4.82 (d, J=13.8 Hz, 1H), 4.80 (d, J=14.1 Hz, 1H), 4.62 (d, J=12.2 Hz, 1H), 4.51 (d, J=10.8 Hz, 1H), 4.43 (s, 2H), 4.21 (d, J=7.7 Hz, 1H), 4.20 (d, J=12.7 Hz, 1H), 4.09 (d, J=12.7 Hz, 1H), 3.91 (s, 2H), 3.76 (dd, J=10.8, 1.9 Hz, 1H), 3.73-3.68 (m, 2H), 3.64 (t, J=8.8 Hz, 1H), 3.59 (t, J=8.8 Hz, 1H), 3.51 (s, 3H), 3.45 (ddd, J=9.3, 4.2, 1.8 Hz, 1H); ¹³C NMR (125 MHz, CDCl₃) δ 142.7, 138.9, 138.6, 138.39, 138.36, 136.1, 128.6, 128.54, 128.46, 128.2, 128.1, 128.0, 127.93, 127.86, 127.8, 127.7, 127.6, 117.7, 113.9, 104.8, 102.7, 84.2, 79.8, 78.1, 75.8, 75.3, 75.1, 73.7, 72.3, 71.1, 69.1, 67.4, 57.2; IR (NaCl) ν 3088, 3064, 3030, 2922, 2860, 1740, 1658, 1606, 1497, 1454, 1058 cm⁻¹; HRMS (ESI) calcd for C₄₂H₄₈O₈Na⁺ (M+Na⁺) 703.3241, found 703.3242.

### Step 2: Preparation of (2R,3R,4S,5R,6R)-3,4-bis(benzyloxy)-2-(benzyloxymethyl)-5-((R)-4-(benzyloxymethyl)-2,5-dihydrofuran-2-yloxy)-6-methoxytetrahydro-2H-pyran

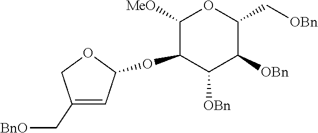 [SMILES: CO[C@@H]1O[C@H](COCc2ccccc2)[C@@H](OCc2ccccc2)[C@H](OCc2ccccc2)[C@H]1O[C@@H]1C=C(COCc2ccccc2)CO1]

(2R,3R,4S,5R,6R)-3, 4-bis(benzyloxy)-2-(benzyloxymethyl)-5-((R)-4-(benzyloxymethyl)-2, 5-dihydrofuran-2-yloxy)-6-methoxytetrahydro-2H-pyran was prepared as a colorless oil (85 mg, 0.13 mmol, 77% yield) by the same general procedure of step 2 above by using the compound obtained in step 1 (118 mg, 0.17 mmol) and CH₂Cl₂ (1.7 mL) containing 1^{st} Grubbs' catalyst (17 mg, 0.017 mmol).

R_{f} 0.47 (Hexane:EtOAc=80:20); [α]²⁵_{D} −3.95 (c 0.62, CH₂Cl₂); 1H NMR (300 MHz, CDCl₃) δ 7.41-7.26 (m, 20H), 6.13 (d, J=3.6 Hz, 1H), 5.78 (s, 1H), 5.03 (d, J=11 Hz, 1H) 4.82 (d, J=10.7 Hz, 1H), 4.71 (d, J=10.8 Hz, 1H), 4.62-4.60 (m, 2H), 4.56-4.43 (m, 5H), 4.21 (d, J=7.9 Hz, 1H), 4.19 (s, 2H), 3.76-3.71 (m, 2H), 3.66 (dd, J=10.5, 5.0 Hz, 1H), 3.55 (s, 3H), 3.46-3.44 (m, 1H); ¹³C NMR (125 MHz, CDCl₃) δ 143.7, 139.0, 138.4, 138.3, 137.9, 128.6, 128.53, 128.47, 128.2, 128.0, 127.98, 127.94, 127.90, 127.87, 127.8, 127.7, 122.7, 110.4, 104.5, 84.0, 80.3, 77.8, 75.5, 75.3, 75.1, 74.3, 73.7, 72.7, 69.2, 65.6, 57.3; IR (NaCl) ν 3088, 3063, 3030, 2923, 2857, 1497, 1359, 1310, 1199, 1076 cm⁻¹; HRMS (ESI) calcd for C₄₀H₄₄O₈Na⁺ (M+Na⁺) 675.2928, found 675.2927.

### Step 3: Preparation of (2R,3R,4R)-4-(benzyloxymethyl)-2-((2R,3R,4S,5R,6R)-4,5-bis(benzyloxy)-6-(benzyloxymethyl)-2-methoxytetrahydro-2H-pyran-3-yloxy)tetrahydrofuran-3,4-diol

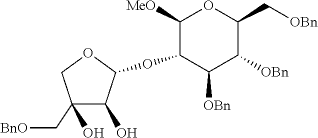 [SMILES: CO[C@H]1O[C@@H](COCc2ccccc2)[C@H](OCc2ccccc2)[C@@H](OCc2ccccc2)[C@@H]1O[C@H]1OC[C@@](O)(COCc2ccccc2)[C@@H]1O]

A target compound was prepared as a yellow oil (26.9 mg, 0.039 mmol, 50% yield) by the same general procedure of step 3 above by using acetone/THF (1:1(v:v), 0.08 mL) containing the compound obtained in step 2 (50.8 mg, 0.08 mmol), 4-methylmorpholine N-oxide (18 mg, 0.16 mmol), OsO₄ solution (4 wt % in H2O, 15.0 μL, 2.7 μmol), and distilled water (0.05 mL, total volume: 0.65 mL, 1.2 M).

R_{f} 0.30 (Hexane:EtOAc=50:50); [α]²⁹_{D} +40.60 (c 1.44, CH₂Cl₂); 1H NMR (300 MHz, CDCl₃) δ 7.37-7.10 (m, 20H), 5.40 (d, J=2.3 Hz, 1H), 4.78 (s, 2H), 4.75 (d, J=10.8 Hz, 1H), 4.62 (d, J=12.2 Hz, 1H), 4.54 (d, J=12.2 Hz, 1H), 4.52-4.48 (m, 3H), 4.24 (d, J=7.7 Hz, 1H), 3.92 (dd, J=2.8, 2.1 Hz, 1H), 3.82 (dd, J=15.1, 10.8 Hz, 2H), 3.74 (dd, J=10.7, 2.0 Hz, 1H), 3.70-3.57 (m, 5H), 3.54 (s, 3H), 3.52-3.48 (m, 2H), 3.44 (ddd, J=9.42, 4.5, 2.1 Hz, 1H), 3.36 (d, J=9.42 Hz, 1H); ¹³C NMR (125 MHz, CDCl₃) δ 138.8, 138.4, 138.2, 137.7, 128.8, 128.6, 128.5, 128.2, 127.99, 127.95, 127.7, 109.4, 104.4, 83.8, 78.8, 78.5, 78.3, 78.1, 75.23, 75.20, 75.18, 74.2, 73.8, 73.7, 72.8, 69.1, 57.2; IR (NaCl) ν 3435, 3089, 3063, 3030, 2926, 1953, 1876, 1812, 1734, 1606, 1586, 1497, 1454, 1361, 1311, 1266, 1214, 1074 cm⁻¹; HRMS (ESI) calcd for C₄₀H₄₆O₁₀Na⁺ (M+Na⁺) 709.2983, found 709.2984.

### <Example 4> Preparation of (2S,3R,4R)-4-(benzyloxymethyl)-2-((2S,3R,4S,5R)-2, 3, 5-tris(benzyloxy)tetrahydro-2H-pyran-4-yloxy)tetrahydrofuran-3, 4-diol

### Step 1: Preparation of (2S,3R,4S,5R)-2, 3, 5-tris(benzyloxy)-4-((S)-1-(2-(benzyloxymethyl)allyloxy)allyloxy)tetrahydro-2H-pyran

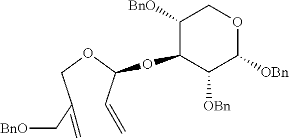 [SMILES: C=C[C@@H](OCC(=C)COCc1ccccc1)O[C@@H]1[C@@H](OCc2ccccc2)[C@@H](OCc2ccccc2)OC[C@H]1OCc1ccccc1]

(2S,3R,4S,5R)-2, 3, 5-tris(benzyloxy)-4-((S)-1-(2-(benzyloxymethyl)allyloxy)allyloxy)tetrahydro-2H-pyran was prepared as a colorless oil (141.3 mg, 0.22 mmol, 74% yield) by the same general procedure of step by using the compound obtained in Preparative Example 2 (134 mg, 0.30 mmol), 2-(benzyloxymethyl)prop-2-en-1-ol (82 mg, 0.44 mmol), Pd₂(dba)₃ (6.4 mg, 7.3 μmol), (R, R)-L1 (16.4 mg, 0.02 mmol), and triethylamine (4.1 μL, 0.03 mmol).

R_{f} 0.69 (Hexane:EtOAc=80:20); [α]²²_{D} +50.1 (c 1.5, CH₂Cl₂); ¹H NMR (300 MHz, CDCl₃) δ 7.40-7.24 (m, 20H), 5.97-5.87 (m, 1H), 5.38-5.32 (m, 3H), 5.14 (d, J=9.3 Hz, 2H), 4.83 (d, J=11.7 Hz, 1H), 4.71-4.40 (m, 8H), 4.25-4.08 (m, 3H), 3.95 (s, 2H), 3.60-3.51 (m, 3H), 3.38 (dd, J=6.0, 3.5 Hz, 1H); ¹³C NMR (75 MHz, CDCl₃) δ 143.0, 138.8, 138.7, 138.3, 137.4, 136.1, 128.6, 128.5, 128.4, 128.1, 128.01, 127.93, 127.87, 127.84, 127.7, 118.0, 113.7, 103.7, 95.2, 80.5, 78.4, 77.1, 73.8, 73.0, 72.2, 71.2, 68.9, 66.8, 60.5; IR (NaCl) ν 3064, 3030, 2880, 1586, 1497 cm⁻¹; HRMS (ESI) calcd for C₄₀H₄₄O₇Na⁺ (M+Na⁺) 659.2979, found 659.2978.

### Step 2: Preparation of (2S,3R,4S,5R)-2,3,5-tris(benzyloxy)-4-((S)-4-(benzyloxymethyl)-2,5-dihydrofuran-2-yloxy)tetrahydro-2H-pyran

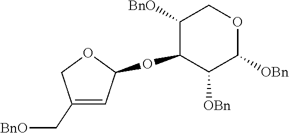 [SMILES: C1=C(COCc2ccccc2)CO[C@H]1O[C@@H]1[C@@H](OCc2ccccc2)[C@@H](OCc2ccccc2)OC[C@H]1OCc1ccccc1]

(2S,3R,4S,5R)-2, 3, 5-tris(benzyloxy)-4-((S)-4-(benzyloxymethyl)-2, 5-dihydrofuran-2-yloxy)tetrahydro-2H-pyran was prepared as a colorless oil (41.7 mg, 0.066 mmol, 83% yield) by the same general procedure of step 2 above by using the compound obtained in step 1 (50.5 mg, 0.08 mmol) and CH₂Cl₂ (0.8 mL) containing 1^{st} Grubbs' catalyst (7.4 mg, 0.009 mmol).

R_{f} 0.65 (Hexane:EtOAc=80:20); [α]²³_{D} +62.4 (c 0.89, CH₂Cl₂); 1H NMR (300 MHz, CDCl₃) δ 7.38-7.21 (m, 20H), 6.20 (d, J=3.9 Hz, 1H), 5.75 (s, 1H), 4.89 (d, J=11.7 Hz, 1H), 4.74-4.45 (m, 10H), 4.22-4.16 (m, 3H), 3.61-3.45 (m, 3H), 3.37 (dd, J=9.6, 3.6 Hz, 1H); ¹³C NMR (75 MHz, CDCl₃) δ 143.7, 138.9, 138.3, 138.0, 137.3, 128.63, 128.56, 128.5, 128.4, 128.1, 128.01, 128.99, 127.9, 127.8, 122.8, 111.2, 95.3, 79.9, 79.6, 74.2, 73.8, 73.1, 72.8, 68.8, 65.7, 60.6; IR (NaCl) ν 3088, 3063, 3030, 2930, 2860, 1586, 1497, cm⁻¹; HRMS (ESI) calcd for C₃₈H₄₀O₇Na⁺ (M+Na⁺) 631.2667, found 631.2666.

### Step 3: Preparation of (2S,3R,4R)-4-(benzyloxymethyl)-2-((2S,3R,4S,5R)-2,3,5-tris(benzyloxy)tetrahydro-2H-pyran-4-yloxy)tetrahydrofuran-3,4-diol

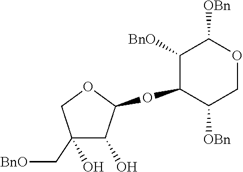 [SMILES: O[C@H]1[C@H](O[C@H]2[C@H](OCc3ccccc3)[C@H](OCc3ccccc3)OC[C@@H]2OCc2ccccc2)OC[C@]1(O)COCc1ccccc1]

A target compound was prepared (36.5 mg, 0.057 mmol, 71% yield) by the same general procedure of step above by using acetone/THF (1:1(v:v), 0.20 mL) containing the compound obtained in step 2 (48.7 mg, 0.08 mmol), 4-methylmorpholine N-oxide (20 mg, 0.17 mmol), OsO₄ solution (4 wt % in H2O, 10.0 μL, 1.5 μmol), and distilled water (0.07 mL, total volume: 0.076 mL, 1.3 M).

m.p. 106-110° C. R_{f} 0.25 (Hexane:EtOAc=50:50); [α]²²_{D} +28.3 (c 1.2, CH₂Cl₂); 1H NMR (300 MHz, CDCl₃) δ 7.39-7.28 (m, 20H), 5.49 (d, J=2.1 Hz, 1H), 4.78-4.45 (m, 9H), 4.13 (t, J=9.3 Hz, 1H), 3.99-3.82 (m, 3H), 3.61-3.57 (m, 3H), 3.46-3.38 (m, 3H), 3.11 (s, 1H), 2.98 (d, J=5.4 Hz, 1H); ¹³C NMR (75 MHz, CDCl₃) 138.5, 138.0, 137.8, 137.3, 128.7, 128.59, 128.57, 128.53, 128.32, 128.23, 128.1, 128.06, 128.01, 127.99, 127.92, 110.1, 95.1, 79.9, 78.8, 78.1, 77.2, 74.2, 73.7, 73.4, 73.0, 72.8, 68.9, 60.3; IR (NaCl) ν 3435, 3063, 3030, 2934, 2884, 1605, 1497, 1454 cm⁻¹; HRMS (ESI) calcd for C₃₈H₄₂O₉Na⁺ (M+Na⁺) 665.2721, found 665.2719.

### <Example 5> Preparation of (2R,3S,4S)-4-(benzyloxymethyl)-2-((2R,3S,4R,5S)-2, 3, 5-tris(benzyloxy)tetrahydro-2H-pyran-4-yloxy)tetrahydrofuran-3, 4-diol

### Step 1: Preparation of (2S,3R,4S,5R)-2, 3, 5-tris(benzyloxy)-4-((R)-1-(2-(benzyloxymethyl)allyloxy)allyloxy)tetrahydro-2H-pyran

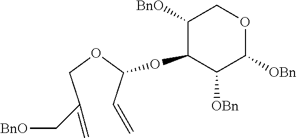 [SMILES: C=C[C@H](OCC(=C)COCc1ccccc1)O[C@@H]1[C@@H](OCc2ccccc2)[C@@H](OCc2ccccc2)OC[C@H]1OCc1ccccc1]

(2S,3R,4S,5R)-2, 3, 5-tris(benzyloxy)-4-((R)-1-(2-(benzyloxymethyl)allyloxy)allyloxy)tetrahydro-2H-pyran was prepared as a colorless oil (210 mg, 0.38 mmol, 90% yield) by the same general procedure of step by using the compound obtained in Preparative Example 2 (200 mg, 0.43 mmol), 2-(benzyloxymethyl)prop-2-en-1-ol (116 mg, 0.65 mmol), Pd₂(dba)₃ (10.0 mg, 0.01 mmol), (S, S)-L1 (22.6 mg, 0.033 mmol), and triethylamine (2.0 μL, 0.02 mmol).

R_{f} 0.55 (Hexane:EtOAc=90:10); [α]²⁷_{D} +55.4 (c=1.7, CH₂Cl₂); 1H NMR (300 MHz, CDCl₃) δ 7.42-7.25 (m, 20H), 5.93 (ddd, J=17.1, 10.5, 5.5 Hz, 1H), 5.40 (d, J=13.4 Hz, 1H), 5.36 (s, 1H), 5.26 (d, J=11.0 Hz, 1H), 5.19 (s, 1H), 5.17 (s, 1H), 4.77 (d, J=3.6 Hz, 1H), 4.74-4.65 (m, 3H), 4.57-4.53 (m, 2H), 4.49-4.45 (m, 3H), 4.28 (d, J=12.7 Hz, 1H), 4.18-4.11 (m, 2H), 3.98 (s, 2H), 3.65-3.51 (m, 3H), 3.46 (dd, J=10.6, 3.5 Hz, 1H); ¹³C NMR (75 MHz, CDCl₃) δ 142.9, 138.6, 138.4, 137.6, 136.2, 128.6, 128.52, 128.48, 128.45, 128.1, 128.0, 127.94, 127.89, 127.8, 127.8 127.6, 118.0, 113.8, 103.7, 96.0, 79.0, 78.9, 77.7, 73.4, 73.2, 72.2, 71.1, 69.0, 66.9, 60.1; IR (NaCl) ν 3088, 3064, 3030, 2931, 2881, 1953, 1875, 1812, 1736, 1658, 1606, 1586, 1497, 1454, 1431, 1408 cm⁻¹; HRMS (ESI) calcd for C₄₀H₄₄O₇Na⁺ (M+Na⁺) 659.2979, found 659.2979.

### Step 2: Preparation of (2S,3R,4S,5R)-2,3,5-tris(benzyloxy)-4-((R)-4-(benzyloxymethyl)-2,5-dihydrofuran-2-yloxy)tetrahydro-2H-pyran

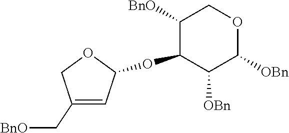 [SMILES: C1=C(COCc2ccccc2)CO[C@@H]1O[C@@H]1[C@@H](OCc2ccccc2)[C@@H](OCc2ccccc2)OC[C@H]1OCc1ccccc1]

(2S,3R,4S,5R)-2,3,5-tris(benzyloxy)-4-((R)-4-(benzyloxymethyl)-2,5-dihydrofuran-2-yloxy)tetrahydro-2H-pyran was prepared as a colorless oil (142 mg, 0.54 mmol, 74% yield) by the same general procedure of step above by using the compound obtained in step 1 (198 mg, 0.31 mmol) and CH₂Cl₂ (3.0 mL) containing 1^{st} Grubbs' catalyst (15 mg, 0.02 mmol).

R_{f} 0.40 (Hexane:EtOAc=90:10); [α]²⁸_{D} +53.1 (c=1.7, CH₂Cl₂); 1H NMR (300 MHz, CDCl₃) δ 7.39-7.22 (m, 20H), 6.18 (d, J=3.8 Hz, 1H), 5.74 (s, 1H), 4.77 (d, J=12.1 Hz, 1H), 4.72-4.65 (m, 4H), 4.59 (d, J=11.7 Hz, 1H), 4.54-4.46 (m, 5H), 4.18-4.12 (m, 3H), 3.60 (d, J=1.8 Hz, 1H), 3.57 (d, J=3.2 Hz, 1H), 3.53-3.47 (m, 1H), 3.36 (dd, J=9.6, 3.6 Hz, 1H); ¹³C NMR (75 MHz, CDCl₃) δ 143.6, 138.9, 138.4, 138.0, 137.5, 128.62, 128.61, 128.5, 128.4, 128.3, 127.97, 127.94, 127.9, 127.6, 122.9, 111.0, 96.1, 79.0, 78.6, 78.5, 74.3, 73.5, 73.4, 72.7, 68.8, 65.6, 60.1; IR (NaCl) ν 3088, 3063, 3030, 2930, 2861, 1954, 1364, 1248, 1202, 1170, 1093, 1028, 942, 835, 735 cm⁻¹; HRMS (ESI) calcd for C₃₈H₄₀O₇Na⁺ (M+Na⁺) 631.2666, found 631.2665.

### Step 3: Preparation of (2R,3S,4S)-4-(benzyloxymethyl)-2-((2R,3S,4R,5S)-2,3,5-tris(benzyloxy)tetrahydro-2H-pyran-4-yloxy)tetrahydrofuran-3,4-diol

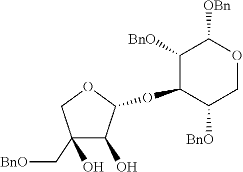 [SMILES: O[C@@H]1[C@@H](O[C@H]2[C@H](OCc3ccccc3)[C@H](OCc3ccccc3)OC[C@@H]2OCc2ccccc2)OC[C@@]1(O)COCc1ccccc1]

A target compound was prepared as a white solid (68 mg, 0.11 mmol, 81% yield) by the same general procedure of step 3 above by using acetone/THF (1:1(v:v), 0.13 mL) containing the compound obtained in step 2 (80.0 mg, 0.13 mmol), 4-methylmorpholine N-oxide (30.8 mg, 0.26 mmol), OsO₄ solution (4 wt % in H2O, 24.0 μL, 4.0 μmol), and distilled water (0.07 mL, total volume: 0.08 mL, 1.6 M).

m.p. 105-108° C. R_{f} 0.30 (Hexane:EtOAc=50:50); [α]²³_{D} +90.2 (c=1.7, CH₂Cl₂); 1H NMR (300 MHz, CDCl₃) 7.37-7.24 (m, 20H), 5.46 (d, J=1.8 Hz, 1H), 4.71-4.63 (m, 3H), 4.58 (d, J=11.6 Hz, 1H), 4.52 (d, J=4.6 Hz, 2H), 4.49 (d, J=8.6 Hz, 1H), 4.45 (d, J=9.2 Hz, 1H), 4.40 (d, J=11.8 Hz, 1H), 4.10 (t, J=9.6 Hz, 1H), 3.94 (d, J=3.0 Hz, 1H), 3.89 (d, J=10.0 Hz, 1H), 3.79 (d, J=10.0 Hz, 1H), 3.61-3.50 (m, 4H), 3.38 (d, J=9.5 Hz, 1H), 3.24 (dd, J=9.6, 3.6 Hz, 1H); ¹³C NMR (75 MHz, CDCl₃) δ 138.4, 138.1, 137.8, 137.3, 128.7, 128.5, 128.4, 128.3, 128.2, 128.11, 128.06, 127.94, 127.91, 127.88, 127.8, 109.7, 95.4, 78.9, 78.8, 78.3, 78.5, 76.4, 74.2, 73.6, 73.1, 73.0, 72.8, 68.8, 59.9; IR (NaCl) ν 3435, 3088, 3063, 3030, 2933, 2883, 1955, 1878, 1813, 1736, 1605, 1586, 1497, 1454, 1366, 1260, 1208, 1173, 1092 cm⁻¹; HRMS (ESI) calcd for C₃₈H₄₂O₉Na⁺ (M+Na⁺) 665.2721, found 665.2721.

### <Example 6> Preparation of (2S,3R,4R)-4-(benzyloxymethyl)-2-((2S,3R,6R)-6-isopropoxy-2-methyltetrahydro-2H-pyran-3-yloxy)tetrahydrofuran-3,4-diol

### Step 1: Preparation of (2S,3R,6R)-3-((S)-1-(2-(benzyloxymethyl)allyloxy)allyloxy)-6-isopropoxy-2-methyltetrahydro-2H-pyran

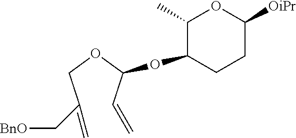 [SMILES: C=C[C@@H](OCC(=C)COCc1ccccc1)O[C@@H]1CC[C@H](OC(C)C)O[C@H]1C]

(2S,3R,6R)-3-(S)-1-(2-(benzyloxymethyl)allyloxy)allyloxy)-6-isopropoxy-2-methyltetrahydro-2H-pyran was prepared as a colorless oil (392 mg, 1.00 mmol, 93% yield) by the same general procedure of step 1 by using (2S,3R,6R)-6-isopropoxy-2-methyl-3-(propa-1,2-dienyloxy)-tetrahydro-2H-pyran (230 mg, 1.08 mmol), 2-(benzyloxymethyl)prop-2-en-1-ol (242 mg, 1.36 mmol), Pd₂(dba)₃ (21.9 mg, 0.024 mmol), (R, R)-L1 (39.4 mg, 34 mmol), and triethylamine (10.0 μL, 0.11 mmol).

R_{f} 0.44 (Hexane:EtOAc=85:15); [α]²⁶_{D} −117.07 (c 0.63, CH₂Cl₂); 1H NMR (300 MHz, CDCl₃) δ 7.35-7.31 (m, 5H), 5.85 (ddd, J=17.1, 10.5, 5.7 Hz, 1H), 5.41 (d, J=17.3 Hz, 1H), 5.29 (d, J=10.5 Hz, 1H), 5.23 (d, J=7.4 Hz, 2H), 4.96 (d, J=4.9 Hz, 1H), 4.84 (d, J=2.8 Hz, 1H), 4.50 (s, 2H), 4.15 (d, J=12.8 Hz, 1H), 4.06-4.02 (m, 3H), 3.94-3.85 (m, 1H), 3.83-3.74 (m, 1H), 3.40-3.32 (m, 1H), 1.95-1.66 (m, 4H), 1.22 (dd, J=10.0, 6.3 Hz, 6H), 1.12 (d, J=6.1 Hz, 3H); ¹³C NMR (125 MHz, CDCl₃) δ 142.8, 138.5, 135.8, 128.6, 127.9, 127.8, 118.7, 114.2, 99.3, 93.9, 75.4, 72.3, 71.3, 67.93, 67.86, 65.9, 29.9, 24.8, 23.6, 21.6, 18.6; IR (NaCl) ν 3065, 3030, 2971, 2932, 2901, 1658, 1497, 1454, 1408, 1379, 1367, 1331, 1229, 1073 cm⁻¹; HRMS (ESI) calcd for C₂₃H₃₄O₅Na⁺ (M+Na⁺) 413.2298, found 413.2298.

### Step 2: Preparation of (2S,3R,6R)-3-((S)-4-(benzyloxymethyl)-2,5-dihydrofuran-2-yloxy)-6-isopropoxy-2-methyltetrahydro-2H-pyran

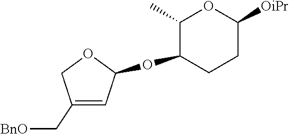 [SMILES: CC(C)O[C@H]1CC[C@@H](O[C@H]2C=C(COCc3ccccc3)CO2)[C@H](C)O1]

(2S,3R,6R)-3-((S)-4-(benzyloxymethyl)-2,5-dihydrofuran-2-yloxy)-6-isopropoxy-2-methyltetrahydro-2H-pyran was prepared as a colorless oil (279 mg, 0.8 mmol, 82.5% yield) by the same general procedure of step 2 above by using the compound obtained in step 1 (378 mg, 0.97 mmol) and CH₂Cl₂ (9 mL) containing 1^{st} Grubbs' catalyst (40 mg, 0.05 mmol).

R_{f} 0.19 (Hexane:EtOAc=90:10); [α]²⁶_{D} −114.9 (c 1.14, CH₂Cl₂); 1H NMR (500 MHz, CDCl₃) δ 7.37-7.29 (m, 5H), 5.92 (d, J=4.0 Hz, 1H), 5.70 (d, J=1.1 Hz, 1H), 4.84 (d, J=2.7 Hz, 1H), 4.67 (d, J=13.5 Hz, 1H), 4.56-4.50 (m, 3H), 4.20 (d, J=4.2 Hz, 2H), 3.91-3.86 (m, 1H), 3.76-3.70 (m, 1H), 3.38-3.34 (m, 1H), 1.93-1.65 (m, 4H), 1.22 (d, J=6.2 Hz, 3H), 1.18 (d, J=6.3 Hz, 3H) 1.11 (d, J=6.1 Hz, 3H); ¹³C NMR (125 MHz, CDCl₃) δ 144.2, 138.0, 128.7, 128.0 127.9, 122.5, 106.7, 93.8, 76.3, 74.0, 72.8, 67.9, 67.7, 65.7, 30.2, 24.9, 23.5, 21.5, 18.3; IR (NaCl) ν 3065, 3031, 2971, 2933, 2901, 2871, 1673, 1558, 1498, 1454, 1380, 1367 cm⁻¹; HRMS (ESI) calcd for C₂₁H₃₀O₅Na⁺ (M+Na⁺) 385.1985, found 385.1983.

### Step 3: Preparation of (2S,3R,4R)-4-(benzyloxymethyl)-2-((2S,3R,6R)-6-isopropoxy-2-methyltetrahydro-2H-pyran-3-yloxy)tetrahydrofuran-3,4-diol

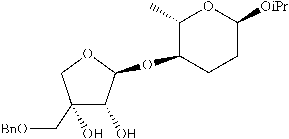 [SMILES: CC(C)O[C@H]1CC[C@@H](O[C@@H]2OC[C@](O)(COCc3ccccc3)[C@H]2O)[C@H](C)O1]

A target compound was prepared as a colorless oil (246.8 mg, 0.62 mmol, 78% yield) by the same general procedure of step 3 above by using acetone/THF (1:1(v:v), 0.8 mL) containing the compound obtained in step 2 (279 mg, 0.8 mmol), 4-methylmorpholine N-oxide (20 mg, 0.17 mmol), OsO₄ solution (4 wt % in H2O, 0.32 mL, 0.05 mmol), and distilled water (0.07 mL, total volume: 10.57 mL, 0.4 M).

R_{f} 0.42 (Hexane:EtOAc=50:50); [α]²⁹_{D} −25.5 (c 0.90, CH₂Cl₂); 1H NMR (500 MHz, CDCl₃) δ 7.32-7.30 (m, 5H), 5.12 (d, J=1.2 Hz, 1H), 4.83 (d, J=1.6 Hz, 1H), 4.58 (d, J=1.4 Hz, 2H), 3.91-3.85 (m, 3H), 3.81 (d, J=10.0 Hz, 1H), 3.68-3.65 (m, 1H), 3.62 (d, J=9.4 Hz, 1H), 3.56 (d, J=9.4 Hz, 1H), 3.29-3.25 (m, 1H), 3.10 (s, 1H), 2.95 (d, J=5.2 Hz, 1H), 1.96-1.93 (m, 1H), 1.78-1.66 (m, 3H), 1.59 (s, 1H), 1.21 (d, J=6.3 Hz, 3H), 1.13 (d, J=6.1 Hz, 3H), 1.10 (d, J=6.2 Hz, 3H); ¹³C NMR (125 MHz, CDCl₃) δ 137.5, 128.8, 128.3, 128.1, 105.2, 94.0, 79.1, 78.2, 75.5, 74.4, 73.9, 73.2, 68.0, 67.5, 29.8, 23.6, 23.3, 21.6, 18.5; IR (NaCl) ν 3407, 3065, 3031, 2971, 2933, 2901, 1737, 1497, 1454, 1379, 1367, 1331, 1231, 1206, 1155 cm⁻¹; HRMS (ESI) calcd for C₂₁H₃₂O₇Na⁺ (M+Na⁺) 419.2040, found 419.2042.

### <Example 7> Preparation of (2R,3S,4S)-4-(benzyloxymethyl)-2-((2S,3R,6R)-6-isopropoxy-2-methyltetrahydro-2H-pyran-3-yloxy)tetrahydrofuran-3, 4-diol

### Step 1: Preparation of (2S,3R,6R)-3-((R)-1-(2-(benzyloxymethyl)allyloxy)allyloxy)-6-isopropoxy-2-methyltetrahydro-2H-pyran

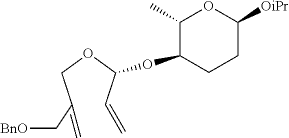 [SMILES: C=C[C@H](OCC(=C)COCc1ccccc1)O[C@@H]1CC[C@H](OC(C)C)O[C@H]1C]

(2S,3R,6R)-3-((R)-1-(2-(benzyloxymethyl)allyloxy)allyloxy)-6-isopropoxy-2-methyltetrahydro-2H-pyran was prepared as a colorless oil (369 mg, 0.95 mmol, 95% yield) by the same general procedure of step 1 by using (2S,3R,6R)-6-isopropoxy-2-methyl-3-(propa-1,2-dienyloxy)-tetrahydro-2H-pyran (214 mg, 1.00 mmol), 2-(benzyloxymethyl)prop-2-en-1-ol (258 mg, 1.50 mmol), Pd₂(dba)₃ (25.0 mg, 0.025 mmol), (S, S)-L1 (35.4 mg, 0.05 mmol), and triethylamine (13.0 μL, 0.1 mmol).

R_{f} 0.23 (Hexane:EtOAc=95:5); [α]²⁹_{D} −66.19 (c 0.76, CH₂Cl₂); H NMR (500 MHz, CDCl₃) δ 7.35-7.27 (m, 5H), 5.82 (ddd, J=17.2, 10.6, 5.5 Hz, 1H), 5.37 (d, J=17.4 Hz, 1H), 5.28 (d, J=11.9 Hz, 1H), 5.22 (d, J=9.6 Hz, 2H), 4.96 (d, J=5.4 Hz, 1H), 4.83 (d, J=2.8 Hz, 1H), 4.50 (s, 2H), 4.11 (d, J=12.8 Hz, 1H), 4.06-4.03 (m, 3H), 3.91-3.86 (m, 1H), 3.80-3.74 (m, 1H), 3.20-3.16 (m, 1H), 1.96-1.84 (m, 2H), 1.77-1.67 (m, 2H), 1.20 (dd, J=8.1, 6.4 Hz, 6H), 1.13 (d, J=6.1 Hz, 3H); ¹³C NMR (125 MHz, CDCl₃) δ 142.8, 138.5, 135.5, 128.6, 127.9, 127.8, 118.6, 114.2, 102.7, 93.9, 78.4, 72.3, 71.2, 68.1, 67.9, 65.6, 30.2, 25.9, 23.6, 21.6, 18.4; IR (NaCl) ν 3065, 3030, 2971, 2932, 1659, 1497, 1454, 1408, 1380, 1367, 1228, 1207, cm⁻¹; HRMS (ESI) calcd for C₂₃H₃₄O₅Na⁺ (M+Na⁺) 413.2298, found 413.2299.

### Step 2: Preparation of (2S,3R,6R)-3-((R)-4-(benzyloxymethyl)-2, 5-dihydrofuran-2-yloxy)-6-isopropoxy-2-methyltetrahydro-2H-pyran

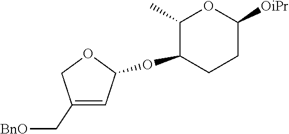 [SMILES: CC(C)O[C@H]1CC[C@@H](O[C@@H]2C=C(COCc3ccccc3)CO2)[C@H](C)O1]

(2S,3R,6R)-3-((R)-4-(benzyloxymethyl)-2,5-dihydrofuran-2-yloxy)-6-isopropoxy-2-methyltetrahydro-2H-pyran was prepared as a colorless oil (276 mg, 0.76 mmol, 86% yield) by the same general procedure of step above by using the compound obtained in step 1 (343 mg, 0.89 mmol) and CH₂Cl₂ (9 mL) containing 1^{st} Grubbs' catalyst (32 mg, 0.04 mmol).

R_{f} 0.18 (Hexane:EtOAc=85:15); [α]²⁹_{D} −83.0 (c 1.24, CH₂Cl₂); 1H NMR (500 MHz, CDCl₃) δ 7.37-7.30 (m, 5H), 5.89 (d, J=4 Hz, 1H), 5.73 (s, 1H), 4.83 (s, 1H), 4.71 (d, J=13.5 Hz, 1H), 4.55-4.50 (m, 3H), 4.19 (d, J=6.6 Hz, 2H), 3.90-3.85 (m, 1H), 3.76-3.71 (m, 1H), 3.25-3.20 (m, 1H), 2.00-1.96 (m, 1H), 1.93-1.84 (m, 1H), 1.76-1.73 (m, 2H), 1.21 (d, J=6.3 Hz, 3H), 1.18 (d, J=6.3 Hz, 3H), 1.11 (d, J=6.1 Hz, 3H); ¹³C NMR (125 MHz, CDCl₃) δ 144.3, 138.0, 128.7, 128.0, 127.9, 122.3, 110.4, 93.8, 79.6, 74.3, 72.9, 68.0, 67.7, 65.7, 30.3, 26.7, 23.6, 21.5, 18.3; IR (NaCl) ν 3031, 2970, 2900, 1673, 1497, 1454, 1379, 1367, 1228, 1205, 1155, 1091 cm⁻¹; HRMS (ESI) calcd for C₂₁H₃₀O₅Na⁺ (M+Na⁺) 385.1985, found 385.1987.

### Step 3: Preparation of (2R,3S,4S)-4-(benzyloxymethyl)-2-((2S,3R,6R)-6-isopropoxy-2-methyltetrahydro-2H-pyran-3-yloxy)tetrahydrofuran-3,4-diol

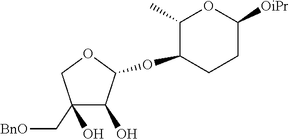 [SMILES: CC(C)O[C@H]1CC[C@@H](O[C@H]2OC[C@@](O)(COCc3ccccc3)[C@@H]2O)[C@H](C)O1]

A target compound was prepared as a colorless oil (127 mg, 0.32 mmol, 70% yield) by the same general procedure of step 3 above by using acetone/THF (1:1(v:v), 0.4 mL) containing the compound obtained in step 2 (150 mg, 0.5 mmol), 4-methylmorpholine N-oxide (120 mg, 1.02 mmol), OsO₄ solution (4 wt % in H₂O, 0.075 mL, 0.014 mmol), and distilled water (0.34 mL, total volume: 0.35 mL, 1.4 M).

R_{f} 0.40 (Hexane:EtOAc=50:50); [α]²⁹_{D} −139.6 (c 0.64, CH₂Cl₂); 1H NMR (500 MHz, CDCl₃) δ 7.38-7.30 (m, 5H), 5.06 (d, J=1.8 Hz, 1H), 4.81 (s, 1H), 4.58 (s, 2H), 3.90-3.83 (m, 4H), 3.67 (dd, J=9.3, 6.3 Hz, 1H), 3.62 (d, J=9.4 Hz, 1H), 3.56 (d, J=9.4 Hz, 1H), 3.19-3.14 (m, 1H), 3.10 (s, 1H), 2.94 (d, J=5.4 Hz, 1H), 1.90-1.85 (m, 1H), 1.77-1.70 (m, 3H), 1.64 (s, 1H), 1.20 (dd, J=8.0, 6.4 Hz, 6H), 1.12 (d, J=6.1 Hz, 3H); ¹³C NMR (125 MHz, CDCl₃) δ 137.5, 128.8, 128.3, 128.1, 110.1, 94.0, 80.0, 78.8, 78.2, 74.1, 73.9, 73.3, 68.0, 67.9, 30.1, 26.4, 23.6, 21.6, 18.2; IR (NaCl) ν 3418, 2970, 2934, 1454, 1380, 1275, 1261, 1228, 1207, 1092 cm⁻¹; HRMS (ESI) calcd for C₂₁H₃₂O₇Na⁺ (M+Na⁺) 419.2040, found 419.2041.

### <Example 8> Preparation of (2R,3R,4R)-4-(benzyloxymethyl)-2-(((2R,3R,4S,5R,6R)-3,4,5,6-tetrakis (benzyloxy)tetrahydro-2H-pyran-2-yl)methoxy)tetrahydrofuran-3,4-diol

### Step 1: Preparation of (2R,3R,4S,5R,6R)-2,3,4,5-tetrakis(benzyloxy)-6-(((S)-1-(2-(benzyloxymethyl)allyloxy)allyloxy)methyl)tetrahydro-2H-pyran

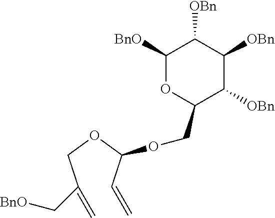 [SMILES: C=C[C@@H](OCC(=C)COCc1ccccc1)OC[C@H]1O[C@@H](OCc2ccccc2)[C@H](OCc2ccccc2)[C@@H](OCc2ccccc2)[C@@H]1OCc1ccccc1]

(2R,3R,4S,5R,6R)-2,3,4,5-tetrakis(benzyloxy)-6-(((S)-1-(2-(benzyloxymethyl)allyloxy)allyloxy)methyl)tetrahydro-2H-pyran was prepared as a colorless oil (283 mg, 0.38 mmol, 87% yield) by the same general procedure of step by using the compound obtained in Preparative Example 3 (200 mg, 0.43 mmol), 2-(benzyloxymethyl)prop-2-en-1-ol (100 mg, 0.56 mmol), Pd₂(dba)₃ (9.0 mg, 0.01 mmol), (R, R)-L1 (17.9 mg, 0.26 mmol), and triethylamine (5.0 μL, 0.036 mmol).

R_{f} 0.57 (Hexane:EtOAc=90:10); [α]²⁷_{D} −8.80 (c 1.2, CH₂Cl₂); 1H NMR (500 MHz, CDCl₃) δ 7.33-7.26 (m, 25H), 5.85 (ddd, J=17.4, 10.6, 4.5 Hz, 1H), 5.44 (d, J=17.4 Hz, 1H), 5.31 (d, J=10.6 Hz, 1H), 5.24 (s, 1H), 5.21 (s, 1H), 5.04 (d, J=4.5 Hz, 1H), 4.95 (d, J=10.9 Hz, 1H), 4.93 (d, J=11.8 Hz, 1H), 4.92 (d, J=11.1 Hz, 1H), 4.86 (d, J=11.0 Hz, 1H), 4.78 (d, J=10.9 Hz, 1H), 4.71 (d, J=10.9 Hz, 1H), 4.65 (d, J=11.9 Hz, 1H), 4.59 (d, J=11.0 Hz, 1H), 4.51 (d, J=7.8 Hz, 1H), 4.48 (s, 2H), 4.17 (d, J=12.8 Hz, 1H), 4.10 (d, J=12.7 Hz, 1H), 4.03 (s, 2H), 3.91 (d, J=10.9 Hz, 1H), 3.67-3.63 (m, 2H), 3.56-3.49 (m, 2H), 3.59-3.46 (ddd, J=10.1, 4.5, 1.5 Hz, 1H); ¹³C NMR (75 MHz, CDCl₃) δ 142.7, 138.8, 138.6, 138.5, 138.3, 137.6, 135.1, 128.63, 128.60, 128.57, 128.56, 128.54, 128.4, 128.15, 128.4, 127.99, 127.96, 127.9, 127.81, 127.76, 119.0, 114.3, 102.7, 101.4, 84.9, 82.5, 78.3, 75.9, 75.2, 75.0, 74.8, 72.3, 71.3, 71.1, 66.1, 64.8; IR (NaCl) ν 3089, 3064, 3031, 2860, 1951, 1874, 1810, 1658, 1606, 1586 cm⁻¹; HRMS (ESI) calcd for C₄₈H₅₂O₈Na⁺ (M+Na⁺) 779.3554, found 779.3555.

### Step 2: Preparation of (2R,3R,4S,5R,6R)-2,3,4,5-tetrakis(benzyloxy)-6-(((S)-4-(benzyloxymethyl)-2,5-dihydrofuran-2-yloxy)methyl)tetrahydro-2H-pyran

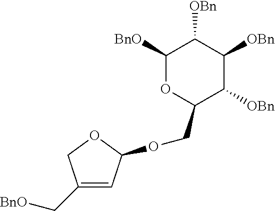 [SMILES: C1=C(COCc2ccccc2)CO[C@H]1OC[C@H]1O[C@@H](OCc2ccccc2)[C@H](OCc2ccccc2)[C@@H](OCc2ccccc2)[C@@H]1OCc1ccccc1]

(2R,3R,4S,5R,6R)-2,3,4,5-tetrakis(benzyloxy)-6-(((S)-4-(benzyloxymethyl)-2,5-dihydrofuran-2-yloxy)methyl)tetrahydro-2H-pyran was prepared as a colorless oil (174 mg, 0.24 mmol, 80% yield) by the same general procedure of step 2 above by using the compound obtained in step 1 (227 mg, 0.3 mmol) and CH₂Cl₂ (5 mL) containing 1^{st} Grubbs' catalyst (11 mg, 0.013 mmol).

R_{f} 0.29 (Hexane:EtOAc=80:20); [α]²⁸_{D} −2.46 (c 1.4, CH₂Cl₂); 1H NMR (500 MHz, CDCl₃) δ 7.38-7.26 (m, 25H), 5.92 (d, J=4 Hz, 1H), 5.73 (s, 1H), 4.98-4.91 (m, 3H), 4.86 (d, J=10.8 Hz, 1H), 4.79 (d, J=11.0 Hz, 1H), 4.71 (d, J=10.8 Hz, 1H), 4.72-4.68 (m, 2H), 4.65 (d, J=11.4 Hz, 1H), 4.57 (d, J=11.7 Hz, 1H), 4.54-4.50 (m, 3H), 4.18 (d, J=3.3 Hz, 2H), 3.98 (dd, J=10.7, 1.7 Hz, 1H), 3.71-3.67 (m, 1H) 3.64 (d, J=9.0 Hz, 1H), 3.57 (t, J=9.5 Hz, 1H), 3.532-3.47 (m, 2H); ¹³C NMR (75 MHz, CDCl₃) δ 144.3, 138.8, 138.6, 138.4, 137.9, 137.7, 128.7, 128.6, 128.6, 128.5, 128.4, 128.1, 128.1, 128.0, 127.9, 127.8, 127.8, 121.9, 109.7, 102.8, 84.9, 82.5, 78.2, 75.9, 75.1, 75.1, 74.8, 72.9, 71.3, 65.6; IR (NaCl) ν 3088, 3063, 3031, 2861, 1739, 1606, 1497, 1361, 1309, 1277, 1207, 1029 cm⁻¹; HRMS (ESI) calcd for C₄₆H₄₈O₈Na⁺ (M+Na⁺) 751.3241, found 751.3242.

### Step 3: Preparation of (2R,3R,4R)-4-(benzyloxymethyl)-2-(((2R,3R,4S,5R,6R)-3,4,5,6-tetrakis(benzyloxy)tetrahydro-2H-pyran-2-yl)methoxy)tetrahydrofuran-3,4-diol

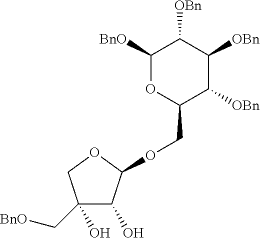 [SMILES: O[C@H]1[C@H](OC[C@H]2O[C@@H](OCc3ccccc3)[C@H](OCc3ccccc3)[C@@H](OCc3ccccc3)[C@@H]2OCc2ccccc2)OC[C@]1(O)COCc1ccccc1]

A target compound was prepared as a colorless oil (127 mg, 0.32 mmol, 70% yield) by the same general procedure of step 3 above by using acetone/THF (1:1(v:v), 0.24 mL) containing the compound obtained in step 2 (174 mg, 0.24 mmol), 4-methylmorpholine N-oxide (56 mg, 0.48 mmol), OsO₄ solution (4 wt % in H2O, 45.0 μL, 7.2 μmol), and distilled water (0.17 mL, total volume: 0.17 mL, 1.4 M).

R_{f} 0.55 (Hexane:EtOAc=50:50); [α]²⁸_{D} −31.7 (c 1.0, CH₂Cl₂); 1H NMR (300 MHz, CDCl₃) δ 7.37-7.30 (m, 25H), 5.03 (d, J=10.9 Hz, 1H), 4.98-4.93 (m, 3H), 4.87 (d, J=10.9 Hz, 1H), 4.81 (d, J=10.9 Hz, 1H), 4.73 (d, J=10.9 Hz, 1H), 4.67 (d, J=11.9 Hz, 1H), 4.59 (d, J=10.9 Hz, 1H), 4.57 (s, 2H), 4.51 (d, J=7.7 Hz, 1H), 3.99-3.89 (m, 4H), 3.69-3.63 (m, 3H), 3.57 (d, J=9.1 Hz, 1H), 3.51 (d, J=7.8 Hz, 1H), 3.47-3.45 (m, 2H), 3.14 (d, J=6.4 Hz, 1H), 2.96 (dd, J=8.3, 5.6 Hz, 1H); ¹³C NMR (75 MHz, CDCl₃) δ 138.7, 138.6, 138.3, 137.6, 137.5, 128.8, 128.7, 128.62, 128.59, 128.55, 128.3, 128.25, 128.2, 128.1, 128.04, 127.99, 127.9, 127.8, 109.1, 102.6, 84.9, 82.6, 79.0, 78.1, 78.0, 75.9, 75.2, 75.1, 74.6, 74.2, 73.9, 73.3, 71.3, 66.7; IR (NaCl) ν 3432, 3089, 3063, 3030, 2921, 2875, 1739, 1606, 1497, 1454, 1398, 1361, 1150 cm⁻¹; HRMS (ESI) calcd for C₄₆H₅₀O₁₀Na⁺ (M+Na⁺), 785.3296, found 785.3298.

### <Example 9> Preparation of (2S,3S,4S)-4-(benzyloxymethyl)-2-(((2R,3R,4S,5R,6R)-3, 4, 5, 6-tetrakis(benzyloxy)tetrahydro-2H-pyran-2-yl)methoxy)tetrahydrofuran-3, 4-diol

### Step 1: Preparation of (2R,3R,4S,5R,6R)-2, 3, 4, 5-tetrakis(benzyloxy)-6-(((R)-1-(2-(benzyloxymethyl)allyloxy)allyloxy)methyl)tetrahydro-2H-pyran

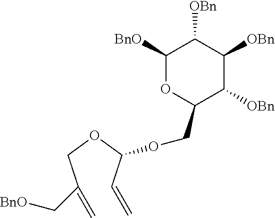 [SMILES: C=C[C@H](OCC(=C)COCc1ccccc1)OC[C@H]1O[C@@H](OCc2ccccc2)[C@H](OCc2ccccc2)[C@@H](OCc2ccccc2)[C@@H]1OCc1ccccc1]

(2R,3R,4S,5R,6R)-2, 3, 4, 5-tetrakis(benzyloxy)-6-(((R)-1-(2-(benzyloxymethyl)allyloxy)allyloxy)methyl)tetrahydro-2H-pyran was prepared as a colorless oil (394 mg, 0.52 mmol, 99% yield) by the same general procedure of step by using the compound obtained in Preparative Example 3 (300 mg, 0.52 mmol), Pd₂(dba)₃ (11.8 mg, 0.013 mmol), (S, S)-L1 (26.8 mg, 0.039 mmol), triethylamine (7.22 μL, 0.052 mmol), and 2-(benzyloxymethyl)prop-2-en-1-ol (138 mg, 0.78 mmol).

R_{f} 0.57 (Hexane:EtOAc=90:10); [α]²⁸_{D} −12.0 (c 1.2, CH₂Cl₂); 1H NMR (300 MHz, CDCl₃) δ 7.37-7.26 (m, 25H), 5.86 (ddd, J=15.3, 10.6, 4.7 Hz, 1H), 5.45 (d, J=17.3 Hz, 1H), 5.31 (d, J=10.5 Hz, 1H), 5.25 (s, 1H), 5.23 (s, 1H), 5.07 (d, J=4.7 Hz, 1H), 4.96-4.92 (m, 3H), 4.87 (d, J=10.9 Hz, 1H), 4.80 (d, J=10.9 Hz, 1H), 4.73 (d, J=10.9 Hz, 1H), 4.66 (d, J=12.0 Hz, 1H), 4.65 (d, J=10.9 Hz, 1H), 4.51 (d, J=7.8 Hz, 1H), 4.49 (s, 2H), 4.23 (d, J=12.6 Hz, 1H), 4.08 (d, J=12.7 Hz, 1H), 4.04 (s, 2H), 3.85-3.74 (m, 2H), 3.69-3.58 (m, 2H), 3.55-3.43 (m, 2H); ¹³C NMR (75 MHz, CDCl₃) δ 142.7, 138.8, 138.6, 138.5, 138.4, 137.7, 135.1, 128.61, 128.58, 128.57, 128.55, 128.4, 128.2, 128.1, 127.9, 119.0, 114.3, 102.7, 101.3, 84.9, 82.6, 78.2, 75.9, 75.2, 75.1, 74.8, 72.3, 71.3, 71.1, 66.5, 63.9; IR (NaCl) ν 3089, 3064, 3031, 2866, 1951, 1873, 1810, 1741, 1658, 1606, 1586, 1497, 1454, 1407, 1361, cm⁻¹; HRMS (ESI) calcd for C₄₈H₅₂O₈Na⁺ (M+Na⁺) 779.3554, found 779.3552.

### Step 2: Preparation of (2R,3R,4S,5R,6R)-2,3,4,5-tetrakis(benzyloxy)-6-(((R)-4-(benzyloxymethyl)-2,5-dihydrofuran-2-yloxy)methyl)tetrahydro-2H-pyran

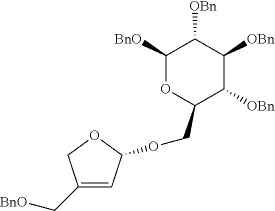 [SMILES: C1=C(COCc2ccccc2)CO[C@@H]1OC[C@H]1O[C@@H](OCc2ccccc2)[C@H](OCc2ccccc2)[C@@H](OCc2ccccc2)[C@@H]1OCc1ccccc1]

(2R,3R,4S,5R,6R)-2,3,4,5-tetrakis(benzyloxy)-6-(((R)-4-(benzyloxymethyl)-2,5-dihydrofuran-2-yloxy)methyl)tetrahydro-2H-pyran was prepared as a colorless oil (273 mg, 0.38 mmol, 77% yield) by the same general procedure of step 2 above by using the compound obtained in step 1 (370 mg, 0.49 mmol) and CH₂Cl₂ (5 mL) containing 1^{st} Grubbs' catalyst (15 mg, 0.018 mmol).

R_{f} 0.18 (Hexane:EtOAc=85:15); [α]²⁸_{D} −15.8 (c 1.3, CH₂Cl₂); 1H NMR (300 MHz, CDCl₃) δ 7.37-7.34 (m, 25H), 6.00 (d, J=4.02 Hz, 1H), 5.82 (d, J=1.1 Hz, 1H), 5.02 (d, J=12.0 Hz, 1H), 5.00 (d, J=10.8 Hz, 1H), 4.97 (d, J=10.8 Hz, 1H), 4.87 (d, J=10.2 Hz, 1H), 4.85 (d, J=11.0 Hz, 1H), 4.77 (d, J=11.0 Hz, 1H), 4.74-4.67 (m, 3H), 4.63-4.58 (m, 2H), 4.55 (s, 2H), 4.21 (s, 2H), 3.93 (dd, J=11.1, 4.0 Hz, 1H), 3.87 (dd, J=11.1, 1.7 Hz, 1H), 3.72-3.69 (m, 2H), 3.59-3.49 (m, 2H); ¹³C NMR (75 MHz, CDCl₃) δ 143.7, 138.8, 138.6, 138.5, 137.9, 137.7, 128.63, 128.60, 128.54, 128.52, 128.49, 128.3, 128.2, 128.1, 128.00, 127.97, 127.94, 127.88, 127.85, 127.8, 127.7, 122.4, 109.6, 102.8, 84.8, 82.4, 77.9, 75.8, 75.1, 75.0, 74.9, 74.5, 72.7, 71.3, 65.8, 65.5; IR (NaCl) ν 3088, 3063, 3030, 2860, 1605, 1497, 1454, 1361, 1308, 1277, 1206, 1150, 1071, 1028, 911 cm⁻¹; HRMS (ESI) calcd for C₄₆H₄₈O₈Na⁺ (M+Na⁺) 751.3241, found 751.3240.

### Step 3: Preparation of (2S,3S,4S)-4-(benzyloxymethyl)-2-(((2R,3R,4S,5R,6R)-3, 4, 5, 6-tetrakis(benzyloxy)tetrahydro-2H-pyran-2-yl)methoxy)tetrahydrofuran-3,4-diol

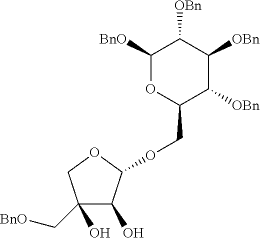 [SMILES: O[C@@H]1[C@@H](OC[C@H]2O[C@@H](OCc3ccccc3)[C@H](OCc3ccccc3)[C@@H](OCc3ccccc3)[C@@H]2OCc2ccccc2)OC[C@@]1(O)COCc1ccccc1]

A target compound was prepared as a yellow solid (212 mg, 0.38 mmol, 88% yield) by the same general procedure of step 3 above by using acetone/THF (1:1(v:v), 0.32 mL) containing the compound obtained in step 2 (230 mg, 0.32 mmol), 4-methylmorpholine N-oxide (74 mg, 0.63 mmol), OsO₄ solution (4 wt % in H2O, 60.0 μL, 60.0 μmol), and distilled water (0.20 mL, total volume: 0.26 mL, 1.2 M).

R_{f} 0.57 (Hexane:EtOAc=50:50); [α]²⁸_{D} +13.00 (c 0.6, CH₂Cl₂); 1H NMR (300 MHz, CDCl₃) δ 7.38-7.26 (m, 25H), 5.10 (d, J=2.0 Hz, 1H), 4.99-4.93 (m, 3H), 4.83 (d, J=10.5 Hz, 1H), 4.81 (d, J=10.5 Hz, 1H), 4.75 (d, J=10.9 Hz, 1H), 4.67 (d, J=11.9 Hz, 1H), 4.61 (d, J=10.5 Hz, 1H), 4.53-4.51 (m, 3H), 4.04-3.99 (m, 2H), 3.91 (d, J=2.5 Hz, 1H), 3.77 (dd, J=11.3, 1.8 Hz, 1H), 3.70-3.451 (m, 5H), 3.43 (ddd, J=9.4, 3.8, 2.2 Hz, 1H), 3.22 (br, 2H); ¹³C NMR (75 MHz, CDCl₃) δ 138.7, 138.5, 138.2, 137.5, 137.4, 128.72, 128.66, 128.63, 128.57, 128.54, 128.3, 128.2, 128.1, 128.0, 127.9, 109.9, 102.7, 84.8, 82.4, 78.9, 78.0, 75.9, 75.2, 75.1, 74.5, 74.2, 73.8, 72.9, 71.4, 67.0; IR (NaCl) ν 3435, 3069, 3054, 3031, 2925, 2874, 2073, 1876, 1810, 1454, 1398, 1361 cm⁻¹; HRMS (ESI) calcd for C₄₆H₅₀O₁₀Na⁺ (M+Na⁺) 785.3296, found 785.3299.

### <Example 10> Preparation of (2R,3R,4S,5R,6R)-2-(benzoyloxymethyl)-6-(((3S,4R,5S)-5-((2R,3R,4S,5R,6R)-4,5-bis(benzyloxy)-6-(benzyloxymethyl)-2-methoxytetrahydro-2H-pyran-3-yloxy)-3,4-dihydroxytetrahydrofuran-3-yl)methoxy)tetrahydro-2H-pyran-3, 4, 5-triyl tribenzoate

### Step 1: Preparation of (2R,3R,4S,5R,6R)-2-(benzoyloxymethyl)-6-(2-(((S)-1-((2R,3R,4S,5R,6R)-4,5-bis(benzyloxy)-6-(benzyloxymethyl)-2-methoxytetrahydro-2H-pyran-3-yloxy)allyloxy)methyl)allyloxy)tetrahydro-2H-pyran-3,4,5-triyl tribenzoate

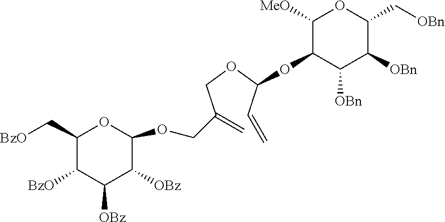

(2R,3R,4S,5R,6R)-2-(benzoyloxymethyl)-6-(2-(((S)-1-((2R,3R,4S,5R,6R)-4,5-bis(benzyloxy)-6-(benzyloxymethyl)-2-methoxytetrahydro-2H-pyran-3-yloxy)allyloxy)methyl)allyloxy)tetrahydro-2H-pyran-3,4,5-triyl tribenzoate was prepared as a white solid (291 mg, 0.25 mmol, 72% yield) by the same general procedure of step 1 by using the compound obtained in Preparative Example 1 (174 mg, 0.346 mmol), Pd₂(dba)₃ (8.33 mg, 0.01 mmol), (R, R)-L1 (12.5 mg, 0.018 mmol), triethylamine (5 μL, 0.035 mmol), and the compound obtained in Preparative Example 4 (291 mg, 0.44 mmol).

m.p. 47-53° C. R_{f} 0.53 (Hexane:EtOAc=50:50); [α]³⁰_{D} +0.1 (c 0.7, CH₂Cl₂); 1H NMR (300 MHz, CDCl₃) δ 8.07-8.04 (m, 2H), 8.00-7.97 (m, 2H), 7.94-7.91 (m, 2H), 7.87-7.84 (m, 2H), 7.55-7.17 (m, 27H), 5.95 (d, J=9.6 Hz, 1H), 5.79-5.61 (m, 1H), 5.73 (d, J=9.5 Hz, 1H), 5.61 (dd, J=9.7, 7.9 Hz, 1H), 5.24 (d, J=17.6 Hz, 1H), 5.21-5.13 (m, 4H), 4.96 (d, J=7.8 Hz, 1H), 4.88 (d, J=10.9 Hz, 1H), 4.82 (d, J=10.7 Hz, 1H), 4.79 (d, J=10.8 Hz, 1H), 4.70-4.51 (m, 5H), 4.42 (d, J=12.9 Hz, 1H), 4.27 (d, J=11.6 Hz, 1H), 4.23-4.15 (m, 2H), 4.13 (d, J=12.6 Hz, 1H), 4.05 (d, J=12.6 Hz, 1H), 3.78 (dd, J=12.7, 2.0 Hz, 1H), 3.73 (dd, J=12.7, 4.4 Hz, 1H), 3.62-3.49 (m, 4H), 3.49 (s, 3H); ¹³C NMR (75 MHz, CDCl₃) δ 166.2, 165.9, 165.3, 165.2, 141.4, 138.5, 138.24, 138.20, 135.1, 133.5, 133.33, 133.25, 129.9, 129.8, 129.7, 129.3, 128.9, 128.5, 128.49, 128.43, 128.0, 127.9, 127.8, 127.73, 127.67, 118.8, 114.8, 103.9, 103.4, 100.0, 84.7, 78.1, 77.8, 75.7, 75.05, 74.95, 73.6, 73.1, 72.3, 72.0, 70.1, 69.9, 69.0, 66.5, 63.3, 56.9; IR (NaCl) ν 3064, 3032, 2922, 2868, 1734, 1602, 1584, 1497, 1452, 1362, 1315, 1267, 1216, 1178, 1095, 1070, 1027, 1002, 937, 853, 803, 737 cm⁻¹; HRMS (ESI) calcd for C₆₉H₆₈O₁₇Na⁺ (M+Na⁺) 1191.4349, found 1191.4347.

### Step 2: Preparation of (2R,3R,4S,5R,6R)-2-(benzoyloxymethyl)-6-(((S)-5-((2R,3R,4S,5R,6R)-4,5-bis(benzyloxy)-6-(benzyloxymethyl)-2-methoxytetrahydro-2H-pyran-3-yloxy)-2,5-dihydrofuran-3-yl)methoxy)tetrahydro-2H-pyran-3,4,5-triyl tribenzoate

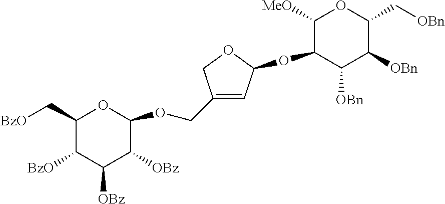

(2R,3R,4S,5R,6R)-2-(benzoyloxymethyl)-6-(((S)-5-((2R,3R,4S,5R,6R)-4,5-bis(benzyloxy)-6-(benzyloxymethyl)-2-methoxytetrahydro-2H-pyran-3-yloxy)-2,5-dihydrofuran-3-yl)methoxy)tetrahydro-2H-pyran-3,4,5-triyl tribenzoate was prepared as a white solid (166 mg, 0.147 mmol, 64% yield, d.r.=1:>20) by the same general procedure of step 2 above by using the compound obtained in step 1 (268 mg, 0.23 mmol) and CH₂Cl₂ (2.3 mL) containing 1^{st} Grubbs' catalyst (12 mg, 0.012 mmol).

m.p. 56-57° C. R_{f} 0.38 (Hexane:EtOAc=70:30); [α]³⁰_{D} +1.6 (c 0.51, CH₂Cl₂); 1H NMR (300 MHz, CDCl₃) δ 8.07-8.04 (m, 2H), 7.98-7.95 (m, 2H), 7.94-7.92 (m, 2H), 7.87-7.84 (m, 2H), 7.56-7.17 (m, 27H), 6.05 (d, J=3.2 Hz, 1H), 5.93 (t, J=9.6 Hz, 1H), 5.72 (t, J=9.7 Hz, 1H), 5.67 (s, 1H), 5.58 (dd, J=9.4, 8.0 Hz, 1H), 4.93-4.87 (m, 2H), 4.81 (d, J=10.7 Hz, 2H), 4.79 (d, J=10.9 Hz, 2H), 4.77-4.53 (m, 6H), 4.37 (d, J=13.6 Hz, 1H), 4.29-4.27 (m, 2H), 4.18-4.14 (m, 1H), 3.77-3.76 (m, 2H), 3.63-3.52 (m, 6H), ¹³C NMR (75 MHz, CDCl₃) δ 166.2, 165.8, 165.2, 165.1, 142.1, 138.6, 138.3, 138.2, 133.5, 133.4, 133.3, 133.27, 129.88, 129.81, 129.6, 129.1, 128.8, 128.51, 128.47, 128.42, 128.38, 128.0, 127.8, 127.7, 127.6, 123.6, 109.9, 103.5, 84.9, 78.6, 78.3, 75.7, 75.1, 74.0, 73.5, 72.9, 72.3, 71.8, 69.7, 69.1, 64.6, 63.1, 57.2; IR (NaCl) ν 3063, 3032, 2863, 1735, 1602, 1584, 1497, 1452, 1361, 1315, 1266, 1198, 1178, 1095, 1070, 1027, 846, 802, 736 cm⁻¹; HRMS (ESI) calcd for C₆₇H₆₄O₁₇Na⁺ (M+Na⁺) 1163.4036, found 1163.4033.

### Step 3: Preparation of (2R,3R,4S,5R,6R)-2-(benzoyloxymethyl)-6-(((3S,4R,5S)-5-((2R,3R,4S,5R,6R)-4, 5-bis(benzyloxy)-6-(benzyloxymethyl)-2-methoxytetrahydro-2H-pyran-3-yloxy)-3, 4-dihydroxytetrahydrofuran-3-yl)methoxy)tetrahydro-2H-pyran-3, 4, 5-triyl tribenzoate

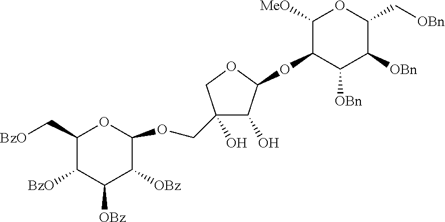

A target compound was prepared as a solid (31 mg, 0.27 mmol, 93% yield) by the same general procedure of step 3 above by using acetone/THF (1:1(v:v), 0.03 mL) containing the compound obtained in step 2 (33 mg, 0.029 mmol), 4-methylmorpholine N-oxide (6.8 mg, 0.06 mmol), OsO₄ solution (4 wt % in H2O, 6 μL, 0.6 μmol), and distilled water (0.014 mL, total volume: 0.02 mL, 1.6 M).

m.p. 142-146° C. R_{f} 0.36 (Hexane:EtOAc=50:50); [α]²⁸_{D} −27.0 (c 0.97, CH₂Cl₂); 1H NMR (500 MHz, CDCl₃) δ 8.07-8.04 (m, 2H), 7.98-7.95 (m, 2H), 7.94-7.92 (m, 2H), 7.87-7.86 (m, 2H), 7.60-7.20 (m, 27H), 5.96 (t, J=9.8 Hz, 1H), 5.70 (t, J=9.8 Hz, 1H), 5.53 (t, J=9.5 Hz, 1H), 5.29 (s, 1H), 4.88 (d, J=12.3 Hz, 2H), 4.82-4.80 (m, 3H), 4.73 (d, J=9.9 Hz, 1H), 4.64 (d, J=12.2 Hz, 1H), 4.58 (d, J=12.3 Hz, 2H), 4.49 (dd, J=7.8, 3.2 Hz, 1H) 4.18 (s, 2H), 3.97-3.92 (m, 3H), 3.79-3.75 (d, J=12.3 Hz, 2H), 3.71 (dd, J=10.9, 4.6 Hz, 2H), 3.60 (br, 3H), 3.54 (br, 1H), 3.47 (br, 4H), 2.88 (d, J=5.1 Hz, 1H); ¹³C NMR (125 MHz, CDCl₃) δ 166.3, 165.9, 165.4, 138.5, 138.4, 138.2, 133.83, 133.78, 133.6, 133.5, 130.1, 130.00, 129.98, 129.94, 129.6, 129.01 128.9, 128.8, 128.7, 128.64, 128.61, 128.55, 128.3, 128.1, 127.98, 127.92, 127.8, 108.8, 103.1, 102.3, 85.3, 78.7, 78.5, 78.4, 77.1, 75.8, 75.2, 75.1, 74.7, 74.3, 73.7, 72.7, 72.6, 72.2, 69.6, 69.0, 63.1, 56.9; IR (NaCl) ν 3475, 3063, 3032, 2928, 1733, 1315, 1267, 1216, 1178, 1095, 1070, 1027, 853, 803, 737, 710 cm⁻¹; HRMS (ESI) calcd for C₆₇H₆₆O₁₉Na⁺ (M+Na⁺) 1197.4091, found 1197.4093.

### <Example 11> Preparation of (2R,3R,4S,5R,6R)-2-(benzoyloxymethyl)-6-(((3S,4R,5R)-5-((2S,3S,4S,5R,6R)-4,5-bis(benzyloxy)-6-(benzyloxymethyl)-2-sec-butyltetrahydro-2H-pyran-3-yloxy)-3,4-dihydroxytetrahydrofuran-3-yl)methoxy)tetrahydro-2H-pyran-3,4,5-triyl tribenzoate

### Step 1: Preparation of (2R,3R,4S,5R,6R)-2-(benzoyloxymethyl)-6-(2-(((R)-1-((2R,3R,4S,5R,6R)-4,5-bis(benzyloxy)-6-benzyloxymethyl)-2-methoxytetrahydro-2H-pyran-3-yloxy)allyloxy)methyl)allyloxy)tetrahydro-2H-pyran-3,4,5-triyl tribenzoate

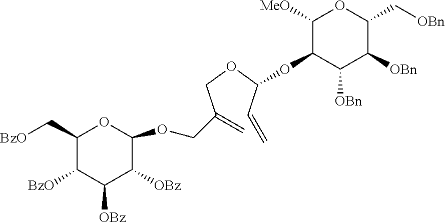

(2R,3R,4S,5R,6R)-2-(benzoyloxymethyl)-6-(2-(((R)-1-((2R,3R,4S,5R,6R)-4,5-bis(benzyloxy)-6-benzyloxymethyl)-2-methoxytetrahydro-2H-pyran-3-yloxy)allyloxy)methyl)allyloxy)tetrahydro-2H-pyran-3,4,5-triyl tribenzoate was prepared as a white solid (670 mg, 0.573 mmol, 96% yield) by the same general procedure of step 1 by using the compound obtained in Preparative Example 1 (300 mg, 0.600 mmol), Pd₂ (dba)₃ (13.6 mg, 0.0149 mmol), (S, S)-L1 (20.6 mg, 0.0298 mmol), triethylamine (8.3 μL, 0.06 mmol), and the compound obtained in Preparative Example 4 (300 mg, 0.6 mmol).

m.p. 49-51° C. R_{f} 0.47 (Hexane:EtOAc=70:30); [α]³⁰_{D} +2.3 (c 0.7, CH₂Cl₂); H NMR (300 MHz, CDCl₃) δ 8.08-8.04 (m, 2H), 8.02-7.98 (m, 2H), 7.95-7.91 (m, 2H), 7.89-7.84 (m, 2H), 7.58-7.17 (m, 27H), 5.91 (t, J=9.6 Hz, 1H), 5.77-5.74 (m, 1H), 5.74 (t, J=9.7 Hz, 1H), 5.63 (dd, J=9.5, 8.0 Hz, 1H), 5.36 (d, J=17.3 Hz, 1H), 5.25-5.20 (m, 2H), 5.12-5.03 (m, 3H), 4.90-4.80 (m, 3H), 4.71-4.52 (m, 5H), 4.27-4.23 (m, 2H), 4.13-4.06 (m, 3H), 3.94 (d, J=12.7 Hz, 1H), 3.84-3.77 (m, 2H), 4.13-4.06 (m, 3H), 3.94 (d, J=12.7 Hz, 1H), 3.84-3.77 (m, 2H), 3.66-3.61 (s, 3H), 3.55-3.52 (m, 1H); ¹³C NMR (75 MHz, CDCl₃) δ 143.9, 138.8, 138.5, 138.3, 138.0, 128.7, 128.6, 128.6, 128.2, 128.0, 128.0, 127.9, 127.8, 127.8, 122.6, 110.5, 103.7, 85.1, 79.4, 78.3, 75.8, 75.2, 75.2, 74.4, 73.7, 72.8, 69.1, 65.6, 57.3, 69.1, 67.4, 57.2; IR (NaCl) ν 3064, 3032, 2923, 2868, 1735, 1602, 1584, 1497, 1452, 1359, 1315, 1266, 1216, 1178, 1094, 1070, 1027, 936, 853, 802 cm⁻¹; HRMS (ESI) calcd for C₆₉H₆₈O₁₇Na⁺ (M+Na⁺) 1191.4349, found 1191.4346.

### Step 2: Preparation of (2R,3R,4S,5R,6R)-2-(benzoyloxymethyl)-6-(((R)-5-((2R,3R,4S,5R,6R)-4,5-bis(benzyloxy)-6-(benzyloxymethyl)-2-methoxytetrahydro-2H-pyran-3-yloxy)-2,5-dihydrofuran-3-)methoxy)tetrahydro-2H-pyran-3,4,5-triyl tribenzoate

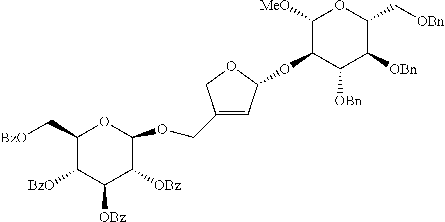

(2R,3R,4S,5R,6R)-2-(benzoyloxymethyl)-6-(((R)-5-((2R,3R,4S,5R,6R)-4,5-bis(benzyloxy)-6-(benzyloxymethyl)-2-methoxytetrahydro-2H-pyran-3-yloxy)-2,5-dihydrofuran-3-yl)methoxy)tetrahydro-2H-pyran-3,4,5-triyl tribenzoate was prepared as a white solid (123 mg, 0.11 mmol, 83% yield, d.r.=>20:1) by the same general procedure of step 2 above by using the compound obtained in step 1 (153 mg, 0.131 mmol) and CH₂Cl₂ (1.3 mL) containing 1^{st} Grubbs' catalyst (10 mg, 0.013 mmol).

m.p. 50-54° C. R_{f} 0.30 (Hexane:EtOAc=70:30); [α]²⁸_{D} +10.1 (c 0.71, CH₂Cl₂); 1H NMR (300 MHz, CDCl₃) δ 8.08-8.06 (m, 2H), 8.01-7.99 (m, 2H), 7.97-7.94 (m, 2H), 7.89-7.87 (m, 2H), 7.58-7.18 (m, 27H), 6.13 (d, J=2.9 Hz, 1H), 5.97 (t, J=9.6 Hz, 1H), 5.77 (s, 1H), 5.75 (t, J=9.7 Hz, 1H), 5.62 (dd, J=9.5, 8.0 Hz, 1H), 5.02 (d, J=10.8 Hz, 1H), 4.93 (d, J=7.8 Hz, 1H), 4.86 (d, J=10.8 Hz, 1H), 4.73-4.39 (m, 12H), 3.82-3.50 (m, 9H); ¹³C NMR (75 MHz, CDCl₃) δ 166.2, 165.9, 165.3, 165.1, 141.9, 138.8, 138.3, 138.2, 133.6, 133.5, 133.4, 133.3, 130.0, 129.9, 129.83, 129.81, 129.6, 129.1, 128.8, 128.5, 128.44, 128.40, 128.37, 128.2, 128.1, 127.8, 127.7, 127.6, 123.9, 110.3, 104.3, 99.8, 80.4, 75.4, 75.1, 75.0, 73.8, 73.6, 72.9, 72.4, 71.8, 69.8, 69.1, 64.2, 63.1, 57.2; IR (NaCl) ν 3063, 3032, 2925, 2865, 1735, 1602, 1585, 1561, 1497, 1452, 1360, 1315, 1267, 1178, 1095, 1070, 1027, 836, 803, 737, 710, 636 cm⁻¹; HRMS (ESI) calcd for C₆₇H₆₄O₁₇Na⁺ (M+Na⁺) 1163.4036, found 1163.4039.

### Step 3: Preparation of (2R,3R,4S,5R,6R)-2-(benzoyloxymethyl)-6-(((3S,4R,5R)-5-((2S,3S,4S,5R,6R)-4,5-bis(benzyloxy)-6-(benzyloxymethyl)-2-sec-butyltetrahydro-2H-pyran-3-yloxy)-3,4-dihydroxytetrahydrofuran-3-yl)methoxy)tetrahydro-2H-pyran-3,4,5-triyl tribenzoate

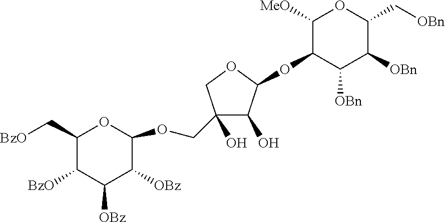

A target compound was prepared as a viscous solid (171 mg, 0.15 mmol, 77% yield) by the same general procedure of step 3 above by using acetone/THF (1:1(v:v), 0.13 mL) containing the compound obtained in step 2 (213.0 mg, 0.19 mmol), 4-methylmorpholine N-oxide (44.2 mg, 0.38 mmol), OsO₄ solution (4 wt % in H2O, 36.0 μL, 6 μmol), and distilled water (0.10 mL, total volume: 0.12 mL, 1.6 M).

m.p. 142-146° C. R_{f} 0.36 (Hexane:EtOAc=50:50); [α]²⁸_{D} +37.3 (c 0.87, CH₂Cl₂); 1H NMR (300 MHz, CDCl₃) δ 8.04-8.03 (m, 2H), 7.96-7.93 (m, 2H), 7.93-7.90 (m, 2H), 7.83-7.80 (m, 2H), 7.58-7.09 (m, 27H), 5.90 (t, J=9.7 Hz, 1H), 5.65 (t, J=9.8 Hz, 1H), 5.46 (dd, J=9.8, 7.9 Hz, 1H), 5.36 (d, J=2.2 Hz, 1H), 4.77-4.69 (m, 5H), 4.61 (d, J=12.2 Hz, 1H), 4.54 (d, J=12.2 Hz, 1H), 4.48 (d, J=10.8 Hz, 1H), 4.42 (dd, J=12.3, 5.3 Hz, 1H), 4.17 (d, J=7.7 Hz, 1H), 4.10 (ddd, J=9.7, 5.2, 3.0 Hz, 1H), 3.88 (d, J=1.9 Hz, 1H), 3.75-3.38 (m, 13H), 1.63 (br, 2H); ¹³C NMR (75 MHz, CDCl₃) δ 166.4, 165.9, 165.6, 165.3, 138.8, 138.3, 138.1, 133.7, 133.5, 130.0, 129.8, 129.5, 129.1, 128.8, 128.6, 128.6, 128.5, 128.4, 128.1, 127.9, 127.7, 127.6, 127.6, 108.8, 104.3, 101.7, 83.5, 78.4, 78.0, 77.9, 75.0, 73.8, 73.6, 73.5, 72.7, 72.6, 72.1, 69.4, 69.0, 62.8, 57.1; IR (NaCl) ν 3475, 3063, 3032, 2932, 2870, 1734, 1602, 1584, 1268, 1216, 1178, cm⁻¹; HRMS (ESI) calcd for C₆₇H₆₆O₁₉Na⁺ (M+Na⁺) 1197.4091, found 1197.4092.

The product of each step of Example 1˜Example 11 and the yield thereof are shown in Table 1.

TABLE 1

| | Step 1 | Step 2 | Step 3 |
|---|---|---|---|
| Exam- | Product compound | Product compound | Product compound |
| ple | (yield) | (yield) | (yield) |
| 1 | 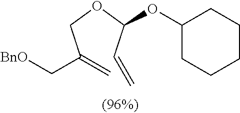 [SMILES: C=C[C@@H](OCC(=C)COCc1ccccc1)OC1CCCCC1] | 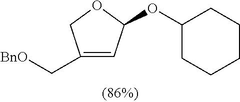 [SMILES: C1=C(COCc2ccccc2)CO[C@H]1OC1CCCCC1] | 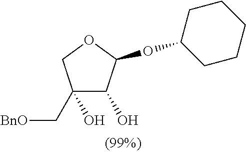 |
| 2 | 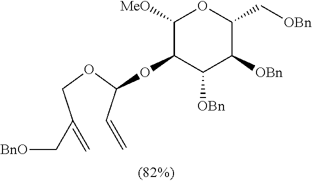 [SMILES: C=C[C@@H](OCC(=C)COCc1ccccc1)O[C@H]1[C@H](OC)O[C@H](COCc2ccccc2)[C@@H](OCc2ccccc2)[C@@H]1OCc1ccccc1] | 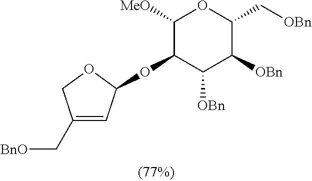 [SMILES: CO[C@@H]1O[C@H](COCc2ccccc2)[C@@H](OCc2ccccc2)[C@H](OCc2ccccc2)[C@H]1O[C@H]1C=C(COCc2ccccc2)CO1] | 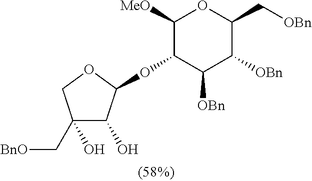 [SMILES: CO[C@H]1O[C@@H](COCc2ccccc2)[C@H](OCc2ccccc2)[C@@H](OCc2ccccc2)[C@@H]1O[C@@H]1OC[C@](O)(COCc2ccccc2)[C@H]1O] |
| 3 | 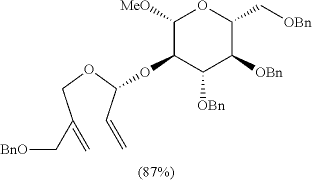 [SMILES: C=C[C@H](OCC(=C)COCc1ccccc1)O[C@H]1[C@H](OC)O[C@H](COCc2ccccc2)[C@@H](OCc2ccccc2)[C@@H]1OCc1ccccc1] | 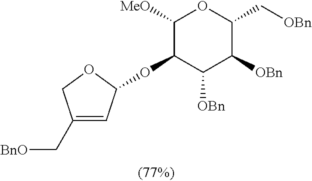 [SMILES: CO[C@@H]1O[C@H](COCc2ccccc2)[C@@H](OCc2ccccc2)[C@H](OCc2ccccc2)[C@H]1O[C@@H]1C=C(COCc2ccccc2)CO1] | 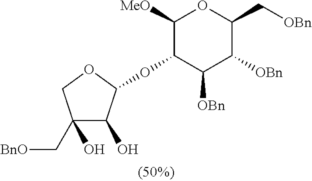 [SMILES: CO[C@H]1O[C@@H](COCc2ccccc2)[C@H](OCc2ccccc2)[C@@H](OCc2ccccc2)[C@@H]1O[C@H]1OC[C@@](O)(COCc2ccccc2)[C@@H]1O] |
| 4 | 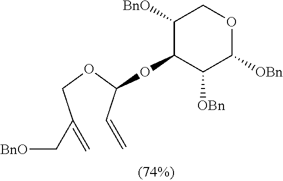 [SMILES: C=C[C@@H](OCC(=C)COCc1ccccc1)O[C@@H]1[C@@H](OCc2ccccc2)[C@@H](OCc2ccccc2)OC[C@H]1OCc1ccccc1] | 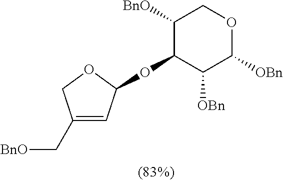 [SMILES: C1=C(COCc2ccccc2)CO[C@H]1O[C@@H]1[C@@H](OCc2ccccc2)[C@@H](OCc2ccccc2)OC[C@H]1OCc1ccccc1] | 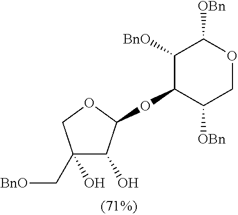 [SMILES: O[C@H]1[C@H](O[C@H]2[C@H](OCc3ccccc3)[C@H](OCc3ccccc3)OC[C@@H]2OCc2ccccc2)OC[C@]1(O)COCc1ccccc1] |
| 5 | 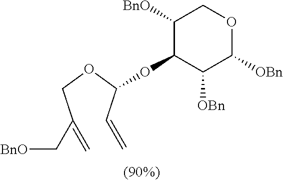 [SMILES: C=C[C@H](OCC(=C)COCc1ccccc1)O[C@@H]1[C@@H](OCc2ccccc2)[C@@H](OCc2ccccc2)OC[C@H]1OCc1ccccc1] | 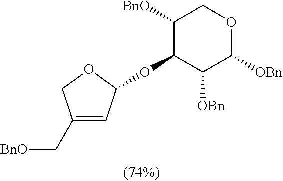 [SMILES: C1=C(COCc2ccccc2)CO[C@@H]1O[C@@H]1[C@@H](OCc2ccccc2)[C@@H](OCc2ccccc2)OC[C@H]1OCc1ccccc1] | 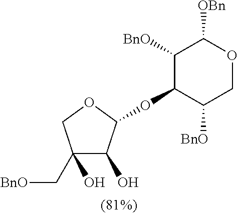 [SMILES: O[C@@H]1[C@@H](O[C@H]2[C@H](OCc3ccccc3)[C@H](OCc3ccccc3)OC[C@@H]2OCc2ccccc2)OC[C@@]1(O)COCc1ccccc1] |
| 6 | 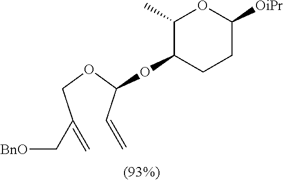 [SMILES: C=C[C@@H](OCC(=C)COCc1ccccc1)O[C@@H]1CC[C@H](OC(C)C)O[C@H]1C] | 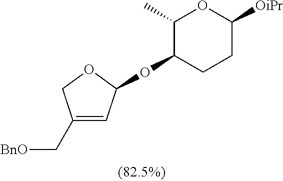 [SMILES: CC(C)O[C@H]1CC[C@@H](O[C@H]2C=C(COCc3ccccc3)CO2)[C@H](C)O1] | 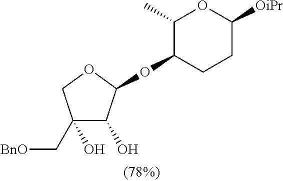 [SMILES: CC(C)O[C@H]1CC[C@@H](O[C@@H]2OC[C@](O)(COCc3ccccc3)[C@H]2O)[C@H](C)O1] |
| 7 | 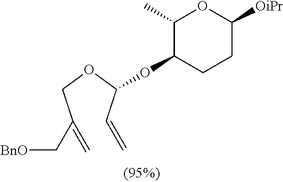 [SMILES: C=C[C@H](OCC(=C)COCc1ccccc1)O[C@@H]1CC[C@H](OC(C)C)O[C@H]1C] | 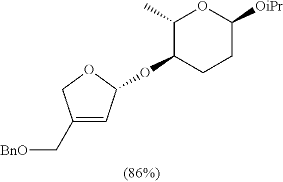 [SMILES: CC(C)O[C@H]1CC[C@@H](O[C@@H]2C=C(COCc3ccccc3)CO2)[C@H](C)O1] | 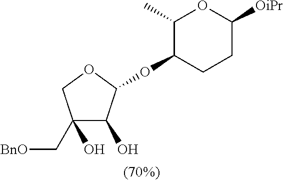 [SMILES: CC(C)O[C@H]1CC[C@@H](O[C@H]2OC[C@@](O)(COCc3ccccc3)[C@@H]2O)[C@H](C)O1] |
| 8 | 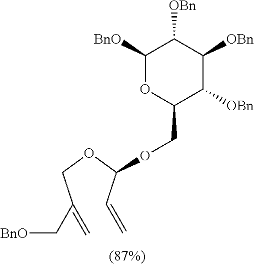 [SMILES: C=C[C@@H](OCC(=C)COCc1ccccc1)OC[C@H]1O[C@@H](OCc2ccccc2)[C@H](OCc2ccccc2)[C@@H](OCc2ccccc2)[C@@H]1OCc1ccccc1] | 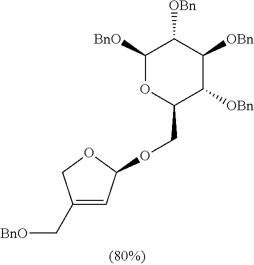 [SMILES: C1=C(COCc2ccccc2)CO[C@H]1OC[C@H]1O[C@@H](OCc2ccccc2)[C@H](OCc2ccccc2)[C@@H](OCc2ccccc2)[C@@H]1OCc1ccccc1] | 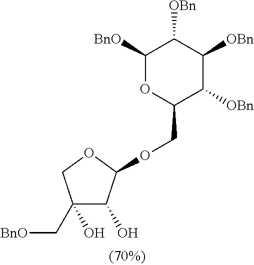 [SMILES: O[C@H]1[C@H](OC[C@H]2O[C@@H](OCc3ccccc3)[C@H](OCc3ccccc3)[C@@H](OCc3ccccc3)[C@@H]2OCc2ccccc2)OC[C@]1(O)COCc1ccccc1] |
| 9 | 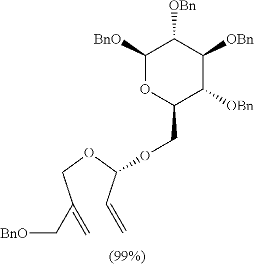 [SMILES: C=C[C@H](OCC(=C)COCc1ccccc1)OC[C@H]1O[C@@H](OCc2ccccc2)[C@H](OCc2ccccc2)[C@@H](OCc2ccccc2)[C@@H]1OCc1ccccc1] | 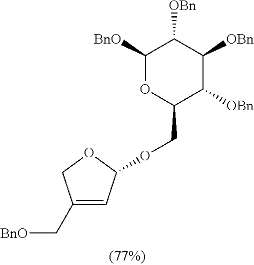 [SMILES: C1=C(COCc2ccccc2)CO[C@@H]1OC[C@H]1O[C@@H](OCc2ccccc2)[C@H](OCc2ccccc2)[C@@H](OCc2ccccc2)[C@@H]1OCc1ccccc1] | 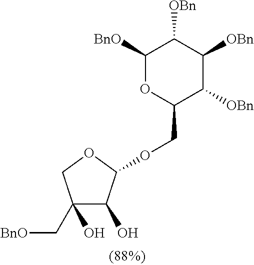 [SMILES: O[C@@H]1[C@@H](OC[C@H]2O[C@@H](OCc3ccccc3)[C@H](OCc3ccccc3)[C@@H](OCc3ccccc3)[C@@H]2OCc2ccccc2)OC[C@@]1(O)COCc1ccccc1] |
| 10 | 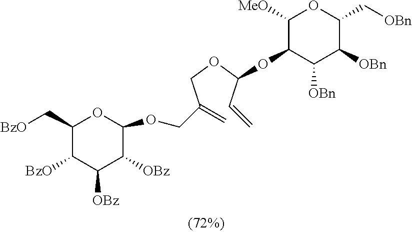 | 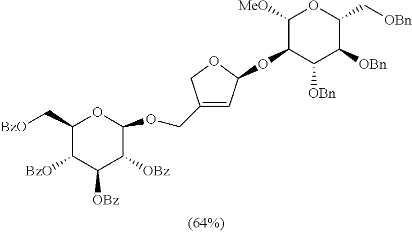 | 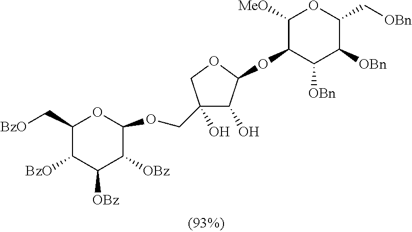 |
| 11 | 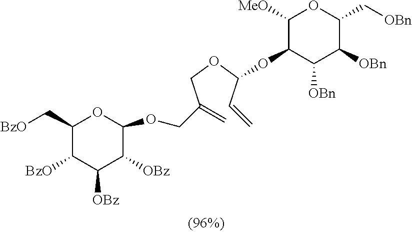 | 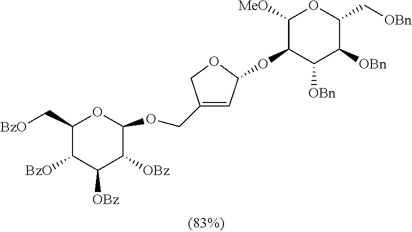 | 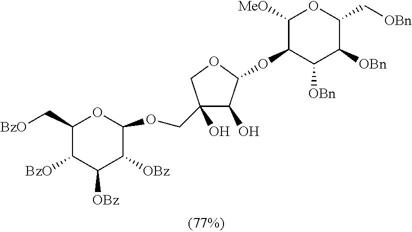 |

As shown in Table 1, acyclic acetal compounds having the stereochemical structure as wanted by the method for the stereoselective preparation were prepared with as high yield as 72˜99% through hydroalkoxylation of step 1 in Examples 1˜11;

Cyclic acetal compounds having the stereochemical structure as wanted by the method for the stereoselective preparation were prepared with as high yield as 64˜86% through ring closing metathesis (RCM) of step 2; and

Apiose derivatives having the stereochemical structure as wanted by the method for the stereoselective preparation were prepared with as high yield as 50˜99.9% through cyclic acetal dehydroxylation of step 3.

Therefore, apiose derivatives, in particular oligosaccharides including monosaccharides, disaccharides, and polysaccharides can be stereoselectively prepared with high yield through simple steps of the method for the stereoselective preparation of apiose derivatives of the present invention.

### <Experimental Example 1> Changes of Yield and Ee Value According to Reaction Condition of Monosaccharide Synthesis

In the preparation method of the invention, cyclic acetal, used as the precursor of a monosaccharide compound, one of apiose derivatives, was synthesized through ring closing metathesis by reacting allylic alcohol compounds and allene compounds. At this time, the changes of yield and ee value according to the reaction condition were investigated by the following experiment as shown in reaction formula 2 below.

Particularly, (propa-1, 2-dienyloxy)cyclohexane was used as the allene starting material and 2-(benzyloxymethyl)prop-2-en-1-ol was used as the allylic alcohol starting material.

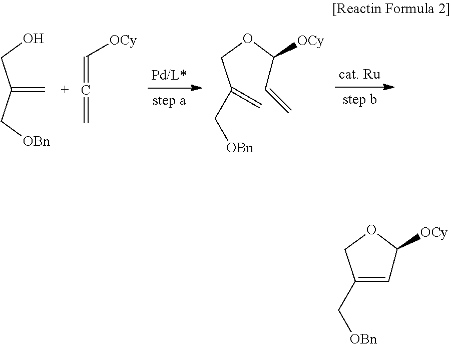

In the reaction formula 2,

L* is a chiral ligand or

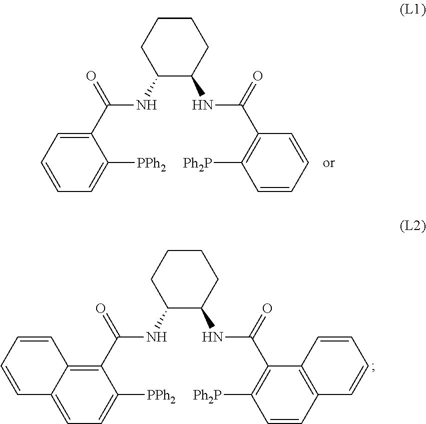

and

cat. Ru is a Ru catalyst selected from the group consisting of

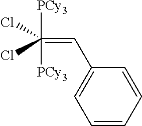

(Ru 1, 1^{st} generation Grubbs catalyst),

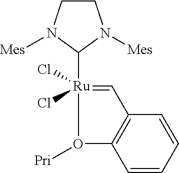

(Ru 2, Hoveyda-Grubbs catalyst 2^{nd} generation) and

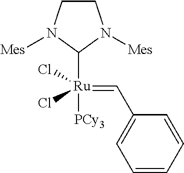

(Ru 3, 2^{nd} generation Grubbs catalyst).

1) Reaction Conditions of Step a

In the reaction formula 2, the catalyst in step a was Pd₂(dba)₃, which was used at the concentration of 2.5 mol %. The dose of a chiral ligand was 5 mol %, and triethylamine (Et₃N) was used as the base necessary herein. The total concentration of the solvent was 0.5 M, and the reaction temperature was 40° C.

In step b, Ru 2 was used as the Ru catalyst (cat. Ru) at the concentration of 5 mol %, and the solvent was CH₂Cl₂ (total concentration 0.05 M). The reaction was induced at room temperature for 24 hours.

The yields of step a and the ee values of step b were measured according to ligand type, base (Et₃N(triethylamine)) equivalent, solvent type, and reaction time in step a. The reaction conditions and the experiment results are shown in Table 2.

TABLE 2

| | Chiral | Et₃N | Reaction | | Step a | Step b |
|---|---|---|---|---|---|---|
| Number | ligand | equivalent | time (h) | Solvent | Yield (%) | Ee (%) |
| 1 | L1 | 1.5 | 0.5 | Toluene | 94 | 96 |
| 2 | L1 | 1.5 | 0.5 | CH₂Cl₂ | 94 | 92 |
| 3 | L1 | 1.5 | 24 | 1,4- | 76 | 87 |
| | | | | dioxane | | |
| 4 | L1 | 0.1 | 0.5 | Toluene | 96 | 97 |
| 5 | L2 | 1.5 | 24 | Toluene | 56 | 88 |

As shown in Table 2, an acyclic acetal compound was produced with the highest yield when Pd₂(dba)₃ (2.5 mol %), the chiral ligand L1 (5 mol %), and triethylamine (0.1 eq) were used in the presence of the nonpolar solvent toluene. In that case, the ee value of the final product after step b was also the highest.

After step b, the ee value of cyclic acetal was 92% or 87% when the more polar solvent dichloromethane (CH₂Cl₂) (Table 2, #2) or 1,4-dioxane (Table 2, #3) was used. When the most nonpolar solvent toluene (Table 2, #1) was used, the best ee value, 96%, was obtained.

Also, when triethylamine was used at the concentration of 0.1 eq, an acyclic acetal compound was obtained with the highest yield (96%) and at that time the ee value was also the highest which was 97%.

When a chiral ligand was used, L1 containing a phenyl group is more advantageous in increasing the yield and the ee value than L2 containing a naphthyl group.

2) Reaction Conditions of Step b

In the reaction formula 2 above, step a was performed under the conditions presented in Table 2, #1.

In step b, the Ru catalyst was used at the concentration of 5 mol %, and the total concentration of the solvent was adjusted to be 0.05 M. The yields of step b were measured according to catalyst type, solvent type, reaction time, and reaction temperature. The reaction conditions and the experiment results are shown in Table 3.

TABLE 3

| | Ru | | Reaction | |
|---|---|---|---|---|
| Number | catalyst | Solvent | temp./time | Yield(%) |
| 1 | Ru 1 | CH₂Cl₂ | 40° C./12 h | N.R. |
| 2 | Ru 3 | CH₂Cl₂ | 40° C./12 h | N.R. |
| 3 | Ru 1 | Toluene | 40° C./12 h | 15 |
| 4 | Ru 3 | Toluene | 40° C./12 h | 6 |
| 5 | Ru 2 | CH₂Cl₂ | RT/36 h | 83 |

In Table 3, N.R. indicates no reaction.

As shown in Table 3, when Ru1 or Ru3 was used as the Ru catalyst (cat. Ru) and dichloromethane (CH₂Cl₂) was used as the solvent, reaction was not induced. When toluene was used as the solvent and reaction was induced by heating, reaction progressed but the progress was poor so that the yield was only up to 20%. In the meantime, when Ru2 was used as the Ru catalyst and dichloromethane was used as the solvent, reaction was induced with producing as high yield as 80%, despite the reaction was induced at room temperature, indicating the yield was increased at least 5 times.

### <Experimental Example 2> Changes of Yield According to Reaction Condition of Disaccharide Synthesis

In the preparation method of the invention, cyclic acetal, used as the precursor of a disaccharide compound was synthesized through ring closing metathesis by reacting allylic alcohol compounds and allene compounds. At this time, the changes of yield according to the reaction condition were investigated by the following experiment as shown in reaction formula 3 below.

Particularly, 2-(benzyloxymethyl)prop-2-en-1-ol was used as the allylic alcohol starting material and the compound having allene groups in different positions was used as the allene compound.

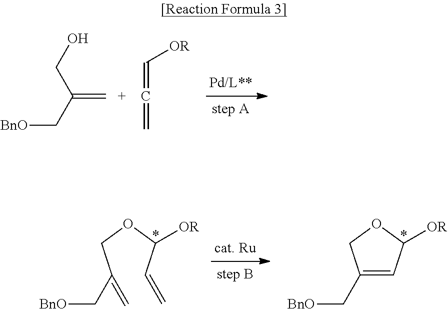

In the reaction formula 3,

L** is

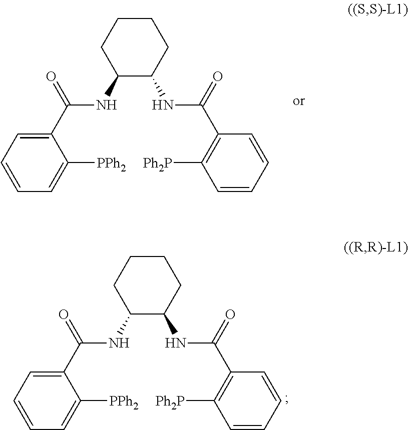

cat. Ru is selected from the group consisting of

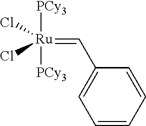

(Ru 1, 1^{st} generation Grubbs catalyst),

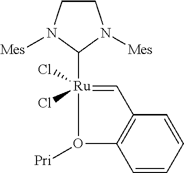

(Ru 2, Hoveyda-Grubbs catalyst 2^{nd} generation), and

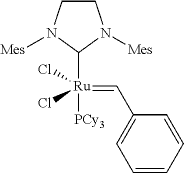

(Ru 3, 2^{nd} generation Grubbs catalyst); and

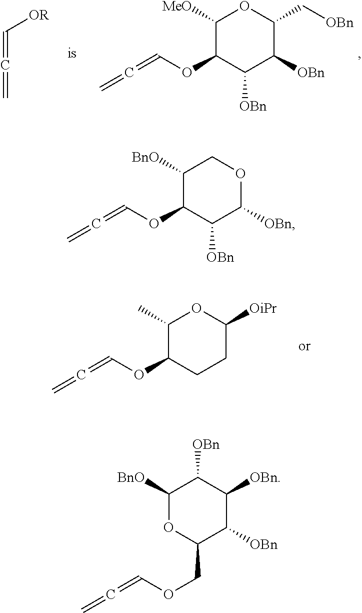

1) Reaction Conditions of Step A

In the reaction formula 3, the catalyst in step A was Pd (dba)₃, which was used at the concentration of 2.5 mol %. Triethylamine (Et₃N, triethylamine, 0.1 eq) was used as the base and toluene was used as the solvent. The total concentration of the solvent was 0.5 M, and the reaction temperature was 40° C.

In step B, Ru1 was used as the Ru catalyst (cat. Ru) at the concentration of 10 mol %, and the solvent was CH₂Cl₂ (total concentration 0.05 M). The reaction was induced at room temperature for 24 hours.

The yields of step A were measured according to the allene starting material of step a, ligand type, and ligand amount. The reaction conditions and the experiment results are shown in Table 4.

TABLE 4

| | | | | | yield | | | |
|---|---|---|---|---|---|---|---|---|
| Num- ber | 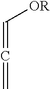 | Chiral ligand | Ligand amount (mol %) | Reaction time (h) | Step A | Step B | Step 2 yield | Final product |
| 1 | 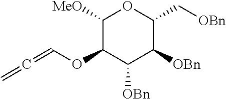 [SMILES: C=C=CO[C@H]1[C@H](OC)O[C@H](COCc2ccccc2)[C@@H](OCc2ccccc2)[C@@H]1OCc1ccccc1] | (R,R)- L1 | 5 | 5 | 82 | 77 | 63 | 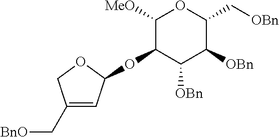 [SMILES: CO[C@@H]1O[C@H](COCc2ccccc2)[C@@H](OCc2ccccc2)[C@H](OCc2ccccc2)[C@H]1O[C@H]1C=C(COCc2ccccc2)CO1] |
| 2 | | (S,S)- L1 | 5 | 3 | 87 | 77 | 67 | 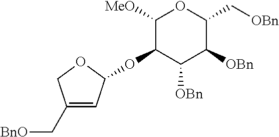 [SMILES: CO[C@@H]1O[C@H](COCc2ccccc2)[C@@H](OCc2ccccc2)[C@H](OCc2ccccc2)[C@H]1O[C@@H]1C=C(COCc2ccccc2)CO1] |
| 3 | 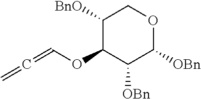 [SMILES: C=C=CO[C@@H]1[C@@H](OCc2ccccc2)[C@@H](OCc2ccccc2)OC[C@H]1OCc1ccccc1] | (R,R)- L1 | 7 .5 | 3 | 74 | 83 | 61 | 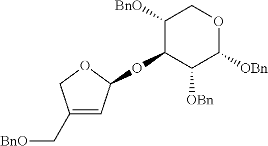 [SMILES: C1=C(COCc2ccccc2)CO[C@H]1O[C@@H]1[C@@H](OCc2ccccc2)[C@@H](OCc2ccccc2)OC[C@H]1OCc1ccccc1] |
| 4 | | (S,S)- L1 | 7.5 | 3 | 90 | 74 | 67 | 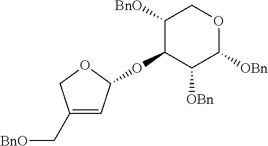 [SMILES: C1=C(COCc2ccccc2)CO[C@@H]1O[C@@H]1[C@@H](OCc2ccccc2)[C@@H](OCc2ccccc2)OC[C@H]1OCc1ccccc1] |
| 5 | 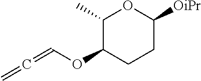 | (R,R)- Ll | 5 | 2 | 93 | 83 | 77 | 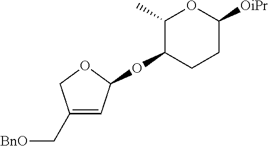 [SMILES: CC(C)O[C@H]1CC[C@@H](O[C@H]2C=C(COCc3ccccc3)CO2)[C@H](C)O1] |
| 6 | | (S,S)- L1 | 5 | 2 | 95 | 86 | 82 | 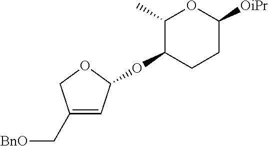 [SMILES: CC(C)O[C@H]1CC[C@@H](O[C@@H]2C=C(COCc3ccccc3)CO2)[C@H](C)O1] |
| 7 | 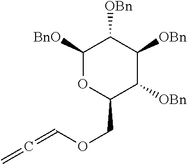 [SMILES: C=C=COC[C@H]1O[C@@H](OCc2ccccc2)[C@H](OCc2ccccc2)[C@@H](OCc2ccccc2)[C@@H]1OCc1ccccc1] | (R,R)- L1 | 7.5 | 8 | 87 | 80 | 70 | 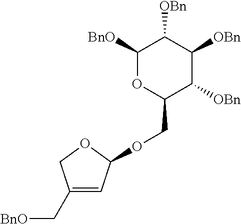 [SMILES: C1=C(COCc2ccccc2)CO[C@H]1OC[C@H]1O[C@@H](OCc2ccccc2)[C@H](OCc2ccccc2)[C@@H](OCc2ccccc2)[C@@H]1OCc1ccccc1] |
| 8 | | (S,S)- L1 | 7.5 | 2 | 99 | 77 | 76 | 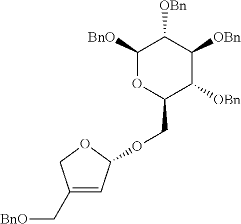 [SMILES: C1=C(COCc2ccccc2)CO[C@@H]1OC[C@H]1O[C@@H](OCc2ccccc2)[C@H](OCc2ccccc2)[C@@H](OCc2ccccc2)[C@@H]1OCc1ccccc1] |

As shown in Table 4, the target compound was synthesized stereoselectively by reacting (D)-glucose having an allene group at C₂, xylose having an allene group at C₃, a deoxysugar compound having an allene group at C₄, and glucopyranoside having an allene group at C₆ as the starting materials with a chiral ligand with the yield of as high as 60-85%.

From the above results, it was confirmed that various disaccharides introduced with apiose at the end of the stereochemical structure could be synthesized by the method for the stereoselective preparation of the present invention with a high yield.

2) Reaction Conditions of Step B

In the reaction formula 3, step A was performed with the conditions presented in Tale 4, #2.

In step B, the Ru catalyst was used at the concentration of 5 mol %. The total concentration of the solvent was 0.5 M, and the reaction was induced at room temperature for 12 hours. The yields of step B were measured with adjusting the kinds of Ru catalyst and the kinds of solvent. The reaction conditions and the experiment results are shown in Table 5.

TABLE 5

| | Ru | | |
|---|---|---|---|
| Number | catalyst | Solvent | yield(%) |
| 1 | Ru 1 | CH₂Cl₂ | 94 |
| 2 | Ru 3 | CH₂Cl₂ | 41 |
| 3 | Ru 2 | CH₂Cl₂ | 19 |
| 4 | Ru 1 | Toluene | 14 |
| 5 | Ru 3 | Toluene | 81 |
| 6 | Ru 2 | Toluene | 83 |

As shown in Table 5, when dichloromethane was used as the Ru catalytic reaction solvent, the highest yield was obtained at 94% when Ru 1 was used. When toluene was used as the solvent, the highest yield was obtained at 83% when Ru 2 was used.

The method for the stereoselective preparation of apiose derivatives of the present invention is efficient in preparing apiose derivatives from allylic alcohol compounds and allene compounds in the presence of a metal catalyst stereoselectively with high yield and high optical purity, regardless of the kinds of substituents of the compound, by using catalytic asymmetric synthesis. The method of the invention can also be used for the preparation of oligosaccharides including monosaccharides, disaccharides, and polysaccharides or various compounds including apiose derivatives because the method can minimize the production of by-products without using an activating group, unlike the conventional method for the preparation of adipose derivatives.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for the stereoselective preparation of apiose derivatives comprising the following steps as shown in reaction formula 1:
preparing the acyclic acetal compound represented by formula 3 by reacting the allylic alcohol compound represented by formula 1 with the allene compound represented by formula 2 in the presence of a metal catalyst (step 1);
preparing the cyclic acetal compound represented by formula 4 by inducing ring closing metathesis of the acyclic acetal compound represented by formula 3 obtained in step 1 in the presence of a metal catalyst (step 2); and
preparing the compound represented by formula 5 by reacting the compound represented by formula 4 obtained in step 2 in the presence of a metal catalyst (step 3). 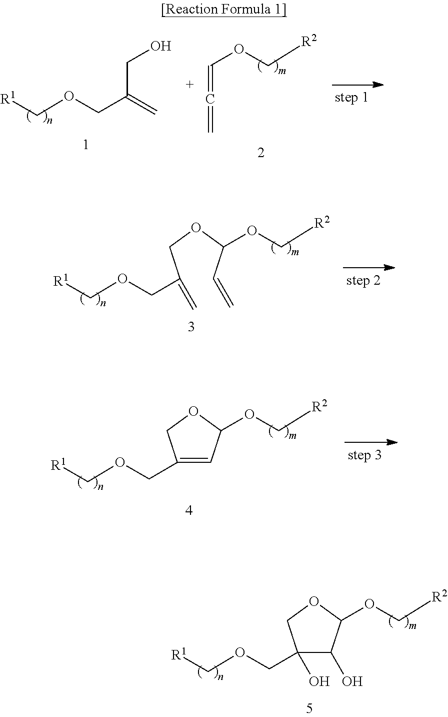
(In the reaction formula 1,
R¹ and R² are independently hydrogen; unsubstituted or substituted straight or branched C₁₋₅ alkyl; unsubstituted or substituted straight or branched C₁₋₅ alkoxy; unsubstituted or substituted 3˜8 membered cycloalkyl; unsubstituted or substituted 3˜8 membered heterocycloalkyl containing one or more O atoms; or unsubstituted or substituted 6˜10 membered aryl;
Wherein, the substituted C₁₋₅ alkyl and C₁₋₅ alkoxy can be substituted with one or more substituents selected from the group consisting of halogen, —OH, unsubstituted 6˜10 membered aryl and unsubstituted 6˜10 membered aryloxy, and the substituted cycloalkyl, heterocycloalkyl, and aryl can be substituted with one or more substituents selected from the group consisting of halogen, —OH, —(CH₂)ₚOR³, unsubstituted straight or branched C₁₋₅ alkyl, unsubstituted straight or branched C₁₋₅ alkoxy, unsubstituted 6˜10 membered aryl, unsubstituted 6˜10 membered aryloxy, unsubstituted 6˜10 membered arylcarbonyl, 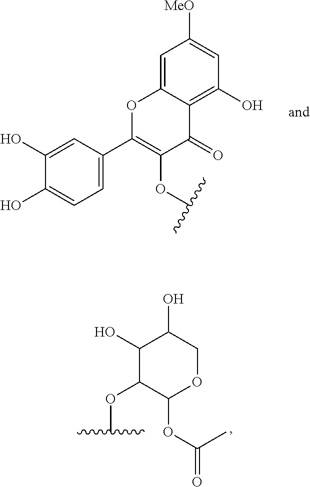 [SMILES: *Oc1c(-c2ccc(O)c(O)c2)oc2cc(OC)cc(O)c2c1=O ; *OC1C(OC(C)=O)OCC(O)C1O]
The said R³ is unsubstituted 6˜10 membered arylC₀₋₂alkyl or unsubstituted 6˜10 membered arylcarbonyl; and
n, m and p are independently 0 or 1).

2. The preparation method according to claim 1, wherein the metal catalyst of step 1 is used at the concentration of 2˜5 mol % by the allene compound represented by formula 2.

3. The preparation method according to claim 1, wherein the metal catalyst of step 2 is used at the concentration of 3˜15 mol % by the acyclic acetal compound represented by formula 3.

4. The preparation method according to claim 1, wherein the metal catalyst of step 3 is used at the concentration of 0.01˜0.1 eq based on 1 eq of the cyclic acetal compound represented by formula 4.

5. The preparation method according to claim 1, wherein the metal catalyst of step 1 and in step 2 is a transition metal complex catalyst.

6. The preparation method according to claim 5, wherein the transition metal is selected from the group consisting of Pd, Ru, Os, Co, Ni, Pt, W, Sn, Al, Mo, and Re.

7. The preparation method according to claim 1, wherein the step 1 is to induce reaction by adding a chiral ligand.

8. The preparation method according to claim 7, wherein the chiral ligand is used at the concentration of 3˜10 mol % by the allene compound represented by formula 2.

9. The preparation method according to claim 1, wherein an organic base or an inorganic base can be additionally used in step 1.

10. The preparation method according to claim 9, wherein the organic base or the inorganic base is used at the concentration of 0.05˜2 eq based on 1 eq of the allene compound represented by formula 2.

11. An intermediate compound for the preparation of apiose derivatives represented by formula 3 below: 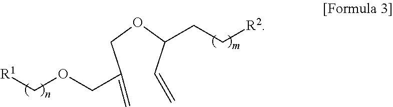
(In the formula 3,
R¹ and R² are independently hydrogen; unsubstituted or substituted straight or branched C₁₋₅ alkyl; unsubstituted or substituted straight or branched C₁₋₅ alkoxy; unsubstituted or substituted 3˜8 membered cycloalkyl; unsubstituted or substituted 3˜8 membered heterocycloalkyl containing one or more O atoms; or unsubstituted or substituted 6˜10 membered aryl;
Wherein, the substituted C₁₋₅ alkyl and C₁₋₅ alkoxy can be substituted with one or more substituents selected from the group consisting of halogen, —OH, unsubstituted 6˜10 membered aryl and unsubstituted 6˜10 membered aryloxy, and the substituted cycloalkyl, heterocycloalkyl, and aryl can be substituted with one or more substituents selected from the group consisting of halogen, —OH, —(CH₂)ₚOR³, unsubstituted straight or branched C>₅ alkyl, unsubstituted straight or branched C₁₋₅ alkoxy, unsubstituted 6˜10 membered aryl, unsubstituted 6˜10 membered aryloxy, unsubstituted 6˜10 membered arylcarbonyl, 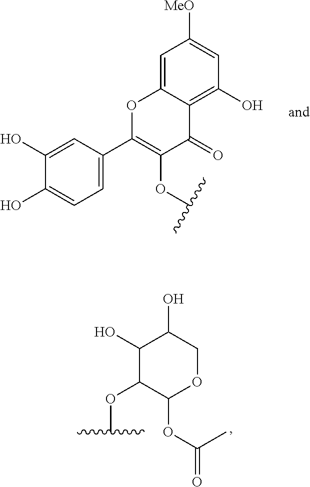 [SMILES: *Oc1c(-c2ccc(O)c(O)c2)oc2cc(OC)cc(O)c2c1=O ; *OC1C(OC(C)=O)OCC(O)C1O]
The said R³ is unsubstituted 6˜10 membered arylC₀₋₂alkyl or unsubstituted 6˜10 membered arylcarbonyl; and
n, m and p are independently 0 or 1).

12. The intermediate compound according to claim 11, wherein:
R¹ and R² are independently unsubstituted or substituted straight or branched C₁₋₃ alkyl; unsubstituted or substituted straight or branched C₁₋₃ alkoxy; unsubstituted or substituted 5 6 membered cycloalkyl; unsubstituted or substituted 5˜6 membered heterocycloalkyl containing one or more O atoms; or unsubstituted or substituted 6˜8 membered aryl;
Wherein, the substituted C₁₋₃ alkyl and C₁₋₃ alkoxy can be substituted with one or more substituents selected from the group consisting of fluoro, chloro, —OH, unsubstituted phenyl, and unsubstituted phenoxy, and the substituted cycloalkyl, heterocycloalkyl, and aryl can be substituted with one or more substituents selected from the group consisting of fluoro, chloro, —OH, —(CH₂)ₚOR³, methyl, ethyl, propyl, isopropyl, methoxy, ethoxy, propoxy, isopropoxy, unsubstituted phenyl, unsubstituted phenoxy, unsubstituted benzoyl, 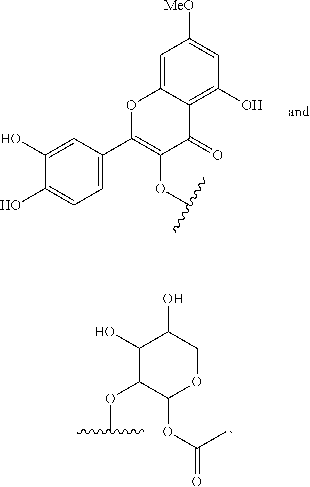 [SMILES: *Oc1c(-c2ccc(O)c(O)c2)oc2cc(OC)cc(O)c2c1=O ; *OC1C(OC(C)=O)OCC(O)C1O]
The said R³ is phenyl, benzyl, or benzoyl; and
n, m and p are independently 0 or 1.

13. The intermediate compound according to claim 11, wherein:
R¹ and R² are independently unsubstituted or substituted cyclohexyl, unsubstituted or substituted tetrahydropyranyl, or unsubstituted or substituted phenyl;
Wherein, the substituted cyclohexyl, tetrahydropyranyl, and phenyl can be substituted with one or more substituents selected from the group consisting of —(CH₂)ₚOR³, methyl, methoxy, isopropoxy, 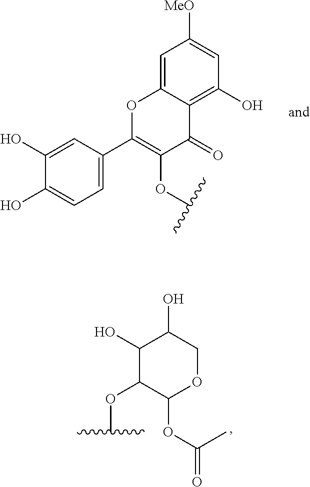 [SMILES: *Oc1c(-c2ccc(O)c(O)c2)oc2cc(OC)cc(O)c2c1=O ; *OC1C(OC(C)=O)OCC(O)C1O]
The said R³ is benzyl or benzoyl; and
n, m and p are independently 0 or 1.

14. An intermediate compound for the preparation of apiose derivatives represented by formula 4 below: 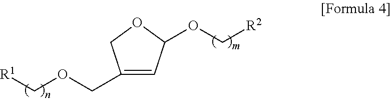
(In the formula 4,
R¹ and R² are independently hydrogen; unsubstituted or substituted straight or branched C₁₋₅ alkyl; unsubstituted or substituted straight or branched C₁₋₅ alkoxy; unsubstituted or substituted 3˜8 membered cycloalkyl; unsubstituted or substituted 3˜8 membered heterocycloalkyl containing one or more 0 atoms; or unsubstituted or substituted 6˜10 membered aryl;
Wherein, the substituted C₁₋₅ alkyl and C₁₋₅ alkoxy can be substituted with one or more substituents selected from the group consisting of halogen, —OH, unsubstituted 6˜10 membered aryl and unsubstituted 6˜10 membered aryloxy, and the substituted cycloalkyl, heterocycloalkyl, and aryl can be substituted with one or more substituents selected from the group consisting of halogen, —OH, —(CH₂)ₚOR³, unsubstituted straight or branched C₁₋₅ alkyl, unsubstituted straight or branched C₁₋₅ alkoxy, unsubstituted 6˜10 membered aryl, unsubstituted 6˜10 membered aryloxy, unsubstituted 6˜10 membered arylcarbonyl, 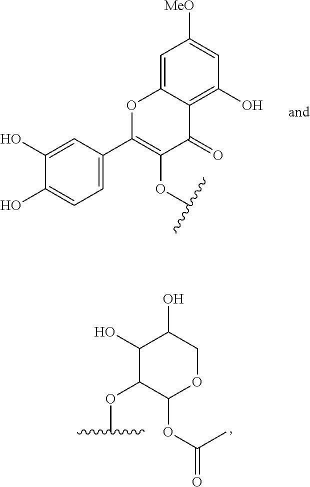 [SMILES: *Oc1c(-c2ccc(O)c(O)c2)oc2cc(OC)cc(O)c2c1=O ; *OC1C(OC(C)=O)OCC(O)C1O]
The said R³ is unsubstituted 6˜10 membered arylC₀₋₂alkyl or unsubstituted 6˜10 membered arylcarbonyl; and
n, m and p are independently 0 or 1).

15. The intermediate compound according to claim 14, wherein:
R¹ and R² are independently unsubstituted or substituted straight or branched C₁₋₃ alkyl; unsubstituted or substituted straight or branched C₁₋₃ alkoxy; unsubstituted or substituted 5˜6 membered cycloalkyl; unsubstituted or substituted 5˜6 membered heterocycloalkyl containing one or more O atoms; or unsubstituted or substituted 6˜8 membered aryl;
Wherein, the substituted C₁₋₃ alkyl and C₁₋₃ alkoxy can be substituted with one or more substituents selected from the group consisting of fluoro, chloro, —OH, unsubstituted phenyl, and unsubstituted phenoxy, and the substituted cycloalkyl, heterocycloalkyl, and aryl can be substituted with one or more substituents selected from the group consisting of fluoro, chloro, —OH, —(CH₂)ₚOR³, methyl, ethyl, propyl, isopropyl, methoxy, ethoxy, propoxy, isopropoxy, unsubstituted phenyl, unsubstituted phenoxy, unsubstituted benzoyl, 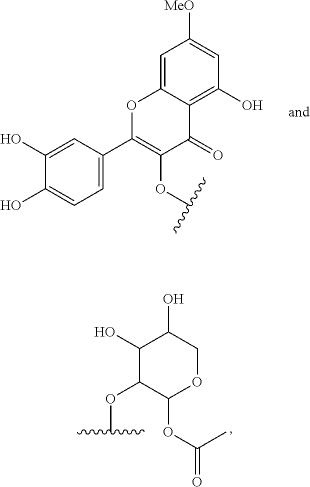 [SMILES: *Oc1c(-c2ccc(O)c(O)c2)oc2cc(OC)cc(O)c2c1=O ; *OC1C(OC(C)=O)OCC(O)C1O]
The said R³ is phenyl or benzoyl; and
n, m and p are independently 0 or 1.

16. The intermediate compound according to claim 14, wherein:
R¹ and R² are independently unsubstituted or substituted cyclohexyl, unsubstituted or substituted tetrahydropyranyl, or unsubstituted or substituted phenyl;
Wherein, the substituted cyclohexyl, tetrahydropyranyl, and phenyl can be substituted with one or more substituents selected from the group consisting of —(CH₂)ₚOR³, methyl, methoxy, isopropoxy, 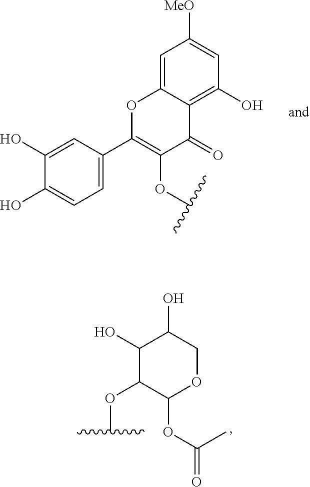 [SMILES: *Oc1c(-c2ccc(O)c(O)c2)oc2cc(OC)cc(O)c2c1=O ; *OC1C(OC(C)=O)OCC(O)C1O]
The said R³ is benzyl or benzoyl; and
n, m and p are independently 0 or 1.
